(19) United States
(12) Patent Application Publication
(10) Pub. No.: US 2023/0149530 A1
(43) Pub. Date: May 18, 2023

(54) INFLUENZA VACCINES

(71) Applicants: DIOSynVax Ltd, Cambridge Cambridgeshire (GB); The Chancellor, Masters and Scholars of the University of Cambridge, Cambridge Cambridgeshire (GB)

(72) Inventors: Jonathan Luke HEENEY, Cambridge Cambridgeshire (GB); Sneha VISHWANATH, Cambridge Cambridgeshire (GB); George CARNELL, Cambridge Cambridgeshire (GB); David WELLS, Cambridge Cambridgeshire (GB); Matteo FERRARI, Cambridge Cambridgeshire (GB)

(21) Appl. No.: 17/916,323

(22) PCT Filed: Apr. 1, 2021
(86) PCT No.: PCT/GB2021/050825

§ 371 Date: Sep. 30, 2022

(30) Foreign Application Priority Data
Apr. 1, 2020 (GB) 2004825.2

(51) Int. Cl. A61K 39/145; C07K 14/005; C12N 15/86

(52) U.S. Cl. CPC A61K 39/145; C07K 14/005; C12N 15/86; A61K 2039/575

(57) ABSTRACT

Polypeptides for use in vaccines against influenza are described. The polypeptides comprise a haemagglutinin subtype 5 (H5) globular head domain, and optionally a haemagglutinin stem domain, with the following amino acid residues at positions 156, 157, 171, 172, and 205 of the head domain: •156: R; •157: P or S, preferably P; •171: D or N; •172: T or A, preferably T; and •205: K or R, preferably K Nucleic acid molecules encoding the polypeptides, vectors, cells, fusion proteins, and pharmaceutical compositions are also described.

This invention relates to nucleic acid molecules, polypeptides, vectors, cells, fusion proteins, pharmaceutical compositions, and their use as vaccines against influenza.

Influenza is a highly contagious respiratory illness caused by the influenza virus infecting the epithelial cells within the upper respiratory tract. The infection is characterised by a sudden onset of high fever, headache, muscle ache and fatigue, sore throat, cough and rhinitis. For the majority of cases, influenza rarely lasts for over a week and is usually restricted to the upper respiratory tract. However, in medically vulnerable people, such as people over 65 years old and people with certain chronic medical conditions, influenza can cause complications and even result in death. There are around 9 million-45 million human infections. WHO estimates that seasonal influenza may result in 290 000-650 000 deaths each year due to respiratory diseases alone. Thus, the development of an effective flu vaccine is critical to the health of millions of people around the world.

The fundamental principal of a vaccine is to prepare the immune system for an encounter with a pathogen. A vaccine triggers the immune system to produce antibodies and T-cell responses, which helps to combat infection. Historically, once a pathogen was isolated and grown, it was either mass produced and killed or attenuated, and used as a vaccine. Later recombinant genes from isolated pathogens were used to generate recombinant proteins that were mixed with adjuvants to stimulate immune responses. More recently the pathogen genes were cloned into vector systems (attenuated bacteria or viral delivery systems) to express and deliver the antigen in vivo. All of these strategies are dependent on pathogens isolated from past outbreaks to prevent future ones. For pathogens which do not change significantly, or slowly, this conventional technology is effective. However, some pathogens, are prone to accelerated mutation rate and previously generated antibodies do not always recognise evolved strains of the same pathogen. New emerging and re-emerging pathogens often hide or disguise their vulnerable antigens from the immune system to escape the immune response.

Influenza is one of the best characterised re-emerging pathogens, and re-emerges each season infecting up to 100 million people worldwide. Influenza is a member of the Orthomyxoviridae family and has a single-stranded negative sense RNA genome. RNA viruses generally have very high mutation rates compared to DNA viruses, because viral RNA polymerases lack the proofreading ability of DNA polymerases. This contributes towards antigenic drift, a continuous process of the accumulation of mutations in the genome of an infectious agent resulting in minor changes in antigens presented to the immune system of the host organism. Changes to antigenic regions of the proteins on the influenza virion result in its evasion of the host immune system and potentially increased pathogenicity and infectiousness. This is one reason why it is difficult to make effective vaccines to prevent influenza. Influenza can undergo antigenic shift, a process wherein there is a dramatic change in the antigens presented on the influenza virus. Gene segments from different subtypes of influenza can reassort and package into a new virion particle containing the genetic information from both of the subtypes. This can result in a virus that has antigenic characteristics not before seen in a human setting, to which we are naïve immunologically. The new quasispecies of the virus can cause a pandemic if no neutralising, or inhibitory antibodies to the new influenza virus are present in the human population.

There are multiple types of influenza viruses, the most common in humans being influenza A, influenza B, and influenza C. Influenza A viruses infect a wide variety of birds and mammals, including humans, horses, marine mammals, pigs, ferrets, and chickens. In their natural reservoirs in aquatic birds and bats, influenza A viruses show minimal evolution and cause unapparent disease; but once they transfer to a different species, influenza A viruses can evolve rapidly as they adapt to the new host, possibly causing pandemics or epidemics of acute respiratory disease in domestic poultry, lower animals and humans. In animals, most influenza A viruses cause mild localized infections of the respiratory and intestinal tract. However, highly pathogenic influenza A strains, such as some within the H5N1 subtype, can cause systemic infections in poultry with spill-over human cases, which can have high mortality rates. Influenza B and C are restricted to infecting humans, with no known animal reservoirs. Influenza B causes epidemic seasonal infections, with similar pathogenicity as influenza A. Influenza C viruses are usually associated with very mild or asymptomatic infections in humans.

At just over 100 years since the devastating 1918 influenza pandemic, there is still no optimal preventative or treatment against influenza A and B. Although they share some degree of similarity with antigen presentation on their surface, the highly heterologous nature of these antigens presents significant challenges in developing vaccines and treatments. During the 2019-2020 seasonal flu epidemic, quadrivalent vaccines were widely distributed. These gave protection against two influenza A viruses and two influenza B viruses. However, to prevent a potential outbreak of influenza in which the virus has rapidly evolved and hence unrecognisable by the host immune system, it is crucial that an influenza vaccine protects against many if not all potential influenza strains.

Influenza A has an outer envelope that is studded with three integral membrane proteins: hemagglutinin (HA); neuraminidase (NA); and matrix ion channel (M2), which overlay a matrix protein (M1). The organisation of influenza B is similar, with HA and NA scattered across the lipid envelope, but with NB and BM2 transmembrane ion channels instead of M2.

Influenza A viruses are subtyped based on their combination of surface glycoproteins (GPs) namely HA and NA. Influenza B viruses, having much less antigenic variation than influenza A, are not. HA and NA are membrane bound envelope GPs, responsible for virus attachment, penetration of the viral particles into the cell, and release of the viral particle from the cell. They are the sources of the major immunodominant epitopes for virus neutralisation and protective immunity. Hence, both HA and NA proteins are considered the most important components for prophylactic influenza vaccines. During HA-mediated entry, binding of the GP to sialic acid-containing receptors on the host cell membrane initiates endocytosis of the virion into the cell. The low pH within the endosome induces a conformational change in HA to expose a hydrophobic region, termed the fusion peptide. The newly exposed fusion peptide then inserts into the endosomal membrane, thereby bringing the viral and endosomal membranes in close contact to allow membrane fusion and entry of the virus into the cytoplasm. This release into the cytoplasm allows viral proteins and RNA molecules to enter the nucleus for viral transcription and subsequent replication. Transcribed, positive sense mRNAs are exported from the nucleus to be translated into viral proteins, and replicated negative sense RNA is exported from the nucleus to re-assemble with the newly synthesised viral proteins to form a progeny virus particle. The virus buds from the apical cell membrane, taking with it host membrane to form a virion capable of infecting another cell.

HA exists as a homo-trimer on the virus surface, forming a cylinder-shaped molecule which projects externally from the virion and forms a type I transmembrane glycoprotein. Each monomer of the HA molecule consists of a single HA0 polypeptide chain with HA1 and HA2 regions linked by two disulphide bridges. Each HA0 polypeptide forms a globular head domain and a stem domain. The globular head domain comprises the most dominant epitopes, while the stem domain has less dominant, but important epitopes for broader antibody recognition. The amino acid sequence of these epitopes determines the binding affinity and specificity towards antibodies. The globular head domain consists of a part of HA1, including a receptor binding domain and an esterase domain, whereas the stem domain consists of parts of HA1 and HA2. Amino acid residues of HA1 that form the globular head domain fold into a motif of eight stranded antiparallel β-sheets which sits in a shallow pocket at the distal tip acting as the receptor binding site which is surrounded by antigenic sites. The remaining parts of the HA1 domain run down to the stem domain mainly comprising β-sheets. HA2 forms the majority of the stem domain and is folded into a helical coiled-coil structure forming the stem backbone. HA2 also contains the hydrophobic region required for membrane fusion, and a long helical chain anchored to the surface membrane and a short cytosolic tail.

There are 18 different HA subtypes and 11 different NA subtypes within influenza A. Theoretically, there are potentially 198 different influenza A subtype combinations, some of which may be virulent in humans and other animals. As a result, there is significant concern that viruses from these subtypes could reassort with human transmissible viruses and initiate the next pandemic. In recent years, avian viruses of the H5, H7, H9, and H10 subtypes have caused zoonotic infections with H5 and H7 viruses often causing severe disease. The highly pathogenic Asian influenza (HPAI) outbreak of H5N1 of 1997 resulted in the killing of the entire domestic poultry population within Hong Kong. This panzootic also resulted in 860 confirmed infections and 454 fatalities in humans, demonstrating the ability of the avian-derived virus to transmit to humans and result in a high mortality rate. This HPAI of the H5N1 subtype frequently re-emerges and is of particular concern because of its 60% mortality rate, and because it continues to evolve and diversify. Although they have less antigenic variation than influenza A viruses, influenza B viruses have recently emerged into two antigenically distinct lineages (B/Victoria/2/1987-like and B/Yamagata/16/1988-like), illustrating the fluidity with which influenza B can evolve, and how it is also now imperative to include viruses of both type A and B in seasonal flu vaccinations.

There is a need to provide improved influenza vaccines that protect against far more influenza strains than current vaccines. In particular, there is a need to provide vaccines against influenza A and B viruses that protect against several influenza A and B variants.

In particular, new vaccine strategies are needed to 1) successfully combat vaccine escape, and, 2) prevent the emergence and spread of new influenza pathogens in the human population. Envisioned herein is the use of large databases of different influenza virus sequences from not only humans, but also animals which are the source of new influenza virus re-assortments which give rise to new human pathogens.

Influenza a H5 Viruses

While most viral genes have been replaced through reassortment yielding many different genotypes, the specific H5 gene has remained present in all influenza A isolates identified since its discovery in 1996. Thus, H5 provides a constant to which the evolving strains of influenza A may be effectively compared. A clade nomenclature system for H5 HA was developed to compare the evolutionary pattern of this gene. Circulating H5N1 viruses are grouped into numerous virus clades based on the characterisation and sequence homology of the HA gene. Clades will have a single common ancestor from which particular genetic changes have arisen. As the viruses within these clades continue to evolve, sub-lineages periodically emerge.

Vaccines against influenza A H5 exist, however either these vaccines are unable to induce a neutralising immune response against the important H5 clades, or the affinity of the antigen to its neutralising antibody is sub-optimal. The computationally optimised broadly reactive antigen (COBRA) Tier 2 vaccine design (Nunez et al, Vaccines, 2020, 38(4):830-839) is developed by consensus sequence alignment techniques using full-length sequences from H5N1 clade 2 infections isolated from both humans and birds. However, this design did not produce haemagglutinin inhibition (HAI) antibodies or protection against newer reassorted viruses across all H5N1 clades and sub-clades that were tested against the vaccine.

There is also a need, therefore, to provide improved vaccines that elicit more broadly neutralising immune responses to influenza A H5 viruses.

The Applicant has identified amino acid sequences and their encoding nucleic acid molecules that induce a broadly neutralising immune response against important H5 clades of influenza A. The Applicant has further identified amino acid sequences and their encoding nucleic acid molecules responsible for stabilising the stem region of the H5 molecule both in the pre-fusion and post-fusion state.

According to the invention there is provided an isolated polypeptide comprising a haemagglutinin subtype 5 (H5) globular head domain, and optionally a haemagglutinin stem domain, with the following amino acid residues at positions 156, 157, 171, 172, and 205 of the head domain:
156: R;
157: P or S, preferably P;
171: D or N;
172: T or A, preferably T; and
205: K or R, preferably K

The applicant has found that such polypeptides elicit broadly neutralising antibody responses to a diverse panel of H5 influenza viruses, including viruses of several different clades.

Optionally a polypeptide of the invention comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:7, 8, 10, 11, 1, or 3.

Optionally a polypeptide of the invention comprises the following amino acid residues at positions 156, 157, 171, 172, and 205 of the head domain:
156: R;
157: P;
171: D;
172: T; and
205: K

Optionally a polypeptide of the invention comprises an amino acid sequence of SEQ ID NO:7 or 8, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:7 or 8 and which has the following amino acid residues at positions corresponding to positions 156, 157, 171, 172, and 205 of SEQ ID NO:7 or 8:
156: R;
157: P;
171: D;
172: T; and
205: K

Optionally a polypeptide of the invention comprises an amino acid sequence of SEQ ID NO:7 (FLU_T3_HA_1) (see Example 4 below).

Such polypeptides are particularly advantageous as they elicit broadly neutralising antibody responses to a diverse panel of H5 influenza viruses, including H5 influenza viruses of clades 2.3.4 and 7.1 arising from the Goose Guangdong (A/Goose/Guangdong/1/1996, GS/GD) lineage, which are currently in circulation in birds and humans.

Optionally a polypeptide of the invention comprises the following amino acid residues at positions 156, 157, 171, 172, and 205 of the head domain:
156: R;
157: P;
171: N;
172: T; and
205: K

Optionally a polypeptide of the invention comprises an amino acid sequence of SEQ ID NO:10 or 11, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:10 or 11 and which has the following amino acid residues at positions corresponding to positions 156, 157, 171, 172, and 205 of SEQ ID NO:0 or 11:
156: R;
157: P;
171: N;
172: T; and
205: K

Optionally a polypeptide of the invention comprises an amino acid sequence of SEQ ID NO:10 (FLU_T3_HA_2) (see Example 5 below).

Such polypeptides are particularly advantageous as they elicit broadly neutralising antibody responses to a diverse panel of H5 influenza viruses, including H5 influenza viruses of GS/GD clades 2.3.4 and 7.1, which are currently in circulation in birds.

Optionally a polypeptide of the invention comprises the following amino acid residues at positions 156, 157, 171, 172, and 205 of the head domain:
156: R;
157: S;
171: N;
172: A; and
205: R

Optionally a polypeptide of the invention comprises an amino acid sequence of SEQ ID NO:1 or 3, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:1 or 3 and which has the following amino acid residues at positions corresponding to positions 156, 157, 171, 172, and 205 of SEQ ID NO:1 or 3:
156: R;
157: S;
171: N;
172: A; and
205: R

Optionally a polypeptide of the invention comprises an amino acid sequence of SEQ ID NO:1 (FLU_T2_HA_1) (see Example 1 below).

Such polypeptides are particularly advantageous as they elicit broadly neutralising antibody responses to a diverse panel of H5 influenza viruses, including viruses of several different GS/GD clades.

Table 1 below summarises differences in amino acid sequence at positions A-E of the influenza haemagglutinin H5 for different embodiments of the invention, and differences at those positions compared with prior art COBRA sequences.

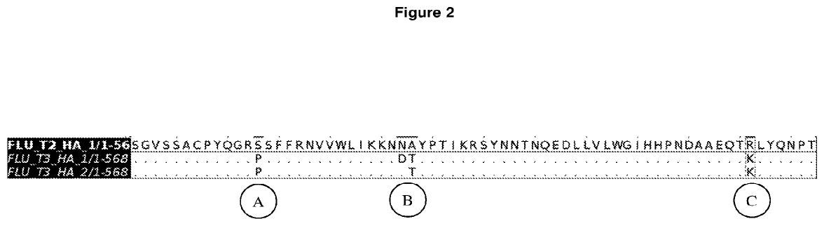

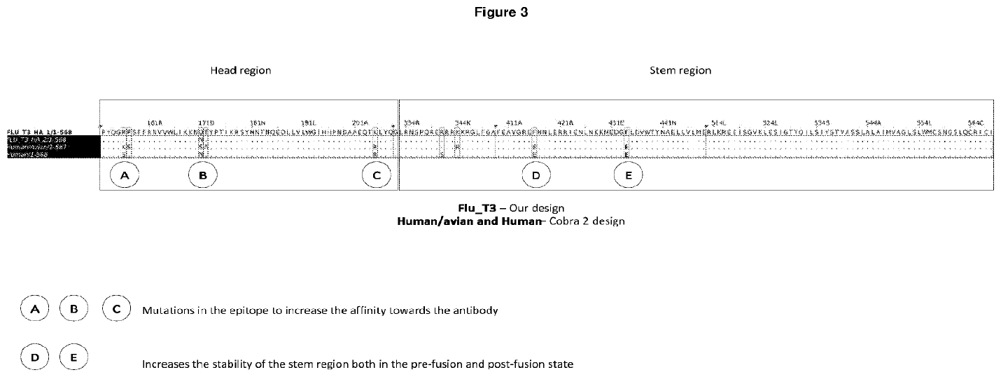

TABLE 1

| | Region | | | | | | | |
|---|---|---|---|---|---|---|---|---|
| | A | | B | | C | D | E | |
| H5 Residue | 156 | 157 | 171 | 172 | 205 | 416 | 434 | Example/FIG. |
| FLU_T2_HA_1 | R | S | N | A | R | F | F | Ex1/FIG. 2 |
| FLU_T3_HA_1 | R | P | D | T | K | F | F | Ex4/FIG. 2 |
| FLU_T3_HA_2 | R | P | N | T | K | F | F | Ex5/FIG. 2 |
| COBRA | K | S | S | A | R | E | E | FIG. 3 |
| (Human/Avian) | | | | | | | | |
| COBRA (Human) | S | P | N | T | R | E | E | FIG. 3 |

A polypeptide of the invention may comprise any suitable haemagglutinin stem domain, including a stem domain of any suitable influenza haemagglutinin subtype, including a non-H5 subtype. Optionally the stem domain is an H5 stem domain.

Optionally a polypeptide of the invention comprises the following amino acid residues at positions 416 and 434 of the stem domain:
416: F; and
434: F

Optionally a polypeptide of the invention is up to 10,000, 9,000, 8,000, 7,000, 6,000, 5,000, 4,000, 3000, 2000, 1500, 1000, 900, 800, 700, 600, 590, 580, 570, 560, 550, 540, 530, 520, 510, 500, 490, 480, 470, 460, 450, 440, 430, 420, 410, 400, 390, 380, 370,360, 350, 340, 330, 320, 310, 300, 290, 280, or 270 amino acid residues in length.

The Applicant has also appreciated that a polypeptide that includes a fragment of the H5 globular head domain with amino acid residues from positions A-C can also elicit an antibody response against H5 influenza viruses. For example, such a polypeptide may be used on its own, or grafted onto other HA subtype heads, or other proteins (for example with a similar folding motif) to generate a suitable antibody response.

Accordingly, there is also provided according to the invention an isolated polypeptide which comprises the following amino acid sequence:

R(P/S)SFFRNVVWLIKKN(D/N)(T/A)YPTIKRSYNNTNQEDLLVLWGIHHPNDAAEQT(K/R) (SEQ ID NO:13), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:13 and which has the following amino acid residues at positions corresponding to positions 1, 2, 16, 17, and 50 of SEQ ID NO:13:
1: R;
2: P or S, preferably P;
16: D or N;
17: T or A, preferably T; and
50: K or R, preferably K.

Optionally a polypeptide of the invention which comprises an amino acid sequence of SEQ ID NO:13, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:13, comprises the following amino acid residues at positions 1, 2, 16, 17, and 50 of the amino acid sequence, or at positions corresponding to positions 1, 2, 16, 17, and 50 of SEQ ID NO:13:
1: R;
2: P;
16: D;
17: T; and
50: K

Optionally a polypeptide of the invention which comprises an amino acid sequence of SEQ ID NO:13, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:13, comprises the following amino acid residues at positions 1, 2, 16, 17, and 50 of the amino acid sequence, or at positions corresponding to positions 1, 2, 16, 17, and 50 of SEQ ID NO:13:
1: R;
2: P;
16: N;
17: T; and
50: K

Optionally a polypeptide of the invention which comprises an amino acid sequence of SEQ ID NO:13, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:13, comprises the following amino acid residues at positions 1, 2, 16, 17, and 50 of the amino acid sequence, or at positions corresponding to positions 1, 2, 16, 17, and 50 of SEQ ID NO:13:
1: R;
2: S;
16: N;
17: A; and
50: R

Optionally a polypeptide of the invention which comprises an amino acid sequence of SEQ ID NO:13, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:13, is up to 570, 560, 550, 500, 450, 400, 350, 300, 250, 200, 150, 100, 90, 80, 70, 60, or 50 amino acid residues in length.

According to the invention there is also provided an isolated polypeptide which comprises an amino acid sequence of any of SEQ ID NOs:5, 9, or 12, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of any of SEQ ID NOs:5, 9, or 12 and which has the following amino acid residues at positions corresponding to positions 148 and 166 of SEQ ID NO:5, 9, or 12:
148: F; and
166: F

The applicant has found that such polypeptides, when forming a stem region of a haemagglutinin molecule, stabilise the stem region in both the pre- and post-fusion state. Such polypeptides may, for example, be provided with an H5 haemagglutinin head domain or a non-H5 head domain.

Optionally a polypeptide of the invention which comprises an amino acid sequence of any of SEQ ID NOs:5, 9, or 12, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of any of SEQ ID NO:5, 9, or 12, is up to 10,000, 9,000, 8,000, 7,000, 6,000, 5,000, 4,000, 3000, 2000, 1500, 1000, 900, 800, 700, 600, 590, 580, 570, 560, 550, 540, 530, 520, 510, 500, 490, 480, 470, 460, 450, 440, 430, 420, 410, 400, 390, 380, 370,360, 350, 340, 330, 320, 310, or 300 amino acid residues in length.

A polypeptide of the invention may include one or more conservative amino acid substitutions. Conservative amino acid substitutions are those substitutions that, when made, least interfere with the properties of the original protein, that is, the structure and especially the function of the protein is conserved and not significantly changed by such substitutions. Examples of conservative substitutions are shown below:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamate or aspartate; or (d) a residue having a bulky side chain, for example, phenylalanine, is substituted for (or by) one not having a side chain, for example, glycine.

There is also provided according to the invention an isolated nucleic acid molecule encoding a polypeptide of the invention, or the complement thereof.

There is also provided according to the invention an isolated nucleic acid molecule comprising a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length to a nucleic acid molecule of the invention encoding polypeptide of the invention, or the complement thereof.

Optionally nucleic acid molecule of the invention comprises a nucleotide sequence of SEQ ID NO:2, 4, or 6, or the complement thereof.

There is also provided according to the invention an isolated nucleic acid molecule comprising a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with SEQ ID NO:2, 4, or 6, or the complement thereof.

The term “broadly neutralising immune response” is used herein in respect of influenza A to include an immune response elicited in a subject that is sufficient to inhibit (i.e. reduce), neutralise or prevent infection, and/or progress of infection, of at least 3 antigenically distinct clades of influenza A. Optionally a broadly neutralising immune response is sufficient to inhibit, neutralise or prevent infection, and/or progress of infection, of different H5 clades of influenza A. Optionally, advantageously the different clades include clades 2.3.4 and/or 7.1.

M2

The extracellular domain of M2 has been identified as being almost invariant across all influenza A strains. This presents as a potential solution to the problem of creating a universal influenza A vaccine that elicits broad-spectrum protection against all influenza A infections.

The Applicant has identified amino acid sequences and their encoding nucleic acid molecules that induce a broadly neutralising immune response against M2 of influenza A.

According to the invention there is provided an isolated polypeptide which comprises an amino acid sequence of SEQ ID NO:14, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:14.

There is also provided according to the invention an isolated nucleic acid molecule, comprising a nucleotide sequence of SEQ ID NO:15, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:15, over its entire length, or the complement thereof.

Neuraminidase

The Applicant has also identified amino acid sequences and their encoding nucleic acid molecules that include epitopes of neuraminidase that are conserved by several different influenza subtypes.

According to the invention there is provided an isolated polypeptide which comprises an amino acid sequence of SEQ ID NO:16, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:16.

According to the invention there is provided an isolated polypeptide which comprises an amino acid sequence of SEQ ID NO:18, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:18.

There is also provided according to the invention an isolated nucleic acid molecule, comprising a nucleotide sequence of SEQ ID NO:17, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:17, over its entire length, or the complement thereof.

There is also provided according to the invention an isolated nucleic acid molecule, comprising a nucleotide sequence of SEQ ID NO:19, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:19, over its entire length, or the complement thereof.

Combination Vaccines

To prevent vaccine escape more effectively, vaccines with a combination of 2 or more (preferably 3 or more) evolutionarily constrained, computationally designed viral antigen targets are provided, each designed to independently give the maximum breadth of vaccine protection. Vaccines of the invention may comprise ancestral antigen based designs of HA, NA and M2, either alone or in combination. Furthermore, combinations of modified HA and NA antigen structures that are not predominantly found to circulate widely as natural combinations in humans are provided (e.g. a group 1 HA combined with a group 2 NA not found to circulate and to co-evolve together, such as H1N1 or H3N2).

Polypeptides or nucleic acid molecules of the invention may be combined in any suitable combination (for example, H5 and/or M2 and/or neuraminidase embodiments of the invention) to provide an influenza vaccine that protects against far more influenza strains than current vaccines. In some embodiments such combination vaccines protect against several influenza A and B variants (especially those embodiments that include M2 embodiments, as M2 is better conserved between influenza A and B).

Optionally a trivalent vaccine combines H5, M2, and neuraminidase embodiments of the invention.

Optionally a nucleic acid vector of the invention comprises:
i) a nucleic acid molecule encoding a polypeptide which comprises an amino acid sequence of SEQ ID NO:7 or 8, or a nucleic acid molecule encoding a polypeptide which comprises an amino acid sequence of SEQ ID NO:10 or 11, or a nucleic acid molecule encoding a polypeptide which comprises an amino acid sequence of SEQ ID NO:1 or 3 (examples of H5 embodiments); and/or
ii) a nucleic acid molecule encoding a polypeptide which comprises an amino acid sequence of SEQ ID NO:14 (examples of M2 embodiments); and/or
iii) a nucleic acid molecule encoding a polypeptide which comprises an amino acid sequence of SEQ ID NO:16, or a nucleic acid molecule encoding a polypeptide which comprises an amino acid sequence of SEQ ID NO:18 (examples of neuraminidase embodiments).

Optionally a vector of the invention further comprises a promoter operably linked to each nucleic acid molecule.

Optionally a vector of the invention is a pEVAC-based vector.

The immune response may be humoral and/or a cellular immune response. A cellular immune response is a response of a cell of the immune system, such as a B-cell, T-cell, macrophage or polymorphonucleocyte, to a stimulus such as an antigen or vaccine. An immune response can include any cell of the body involved in a host defence response, including for example, an epithelial cell that secretes an interferon or a cytokine. An immune response includes, but is not limited to, an innate immune response or inflammation.

Optionally a polypeptide of the invention induces a protective immune response. A protective immune response refers to an immune response that protects a subject from infection or disease (i.e. prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, or antibody production.

Optionally a polypeptide of the invention is able to induce the production of antibodies and/or a T-cell response in a human or non-human animal to which the polypeptide has been administered (either as a polypeptide or, for example, expressed from an administered nucleic acid expression vector).

The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a given gene or protein will possess a relatively high degree of sequence identity when aligned using standard methods. Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids' Research 16:10881-10890, 1988; and Pearson and Lipman, Proc. Natl. Acad. Sci. U.S.A. 85:2444, 1988. Altschul et al., Nature Genet. 6:119-129, 1994. The NCBI Basic Local Alignment Search Tool (BLAST) (Altschul et al., J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx.

Sequence identity between nucleic acid sequences, or between amino acid sequences, can be determined by comparing an alignment of the sequences. When an equivalent position in the compared sequences is occupied by the same nucleotide, or amino acid, then the molecules are identical at that position. Scoring an alignment as a percentage of identity is a function of the number of identical nucleotides or amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4: 29; program available from http://bitincka.com/ledion/matgat), Gap (Needleman & Wunsch, 1970, J. Mol. Biol. 48: 443-453), FASTA (Altschul et al., 1990, J. Mol. Biol. 215: 403-410; program available from http://www.ebi.ac.uk/fasta), Clustal W 2.0 and X 2.0 (Larkin et al., 2007, Bioinformatics 23: 2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

Sequences described herein include reference to an amino acid sequence comprising amino acid residues “at positions corresponding to positions” of another amino acid sequence. Such corresponding positions may be identified, for example, from an alignment of the sequences using a sequence alignment method described herein, or another sequence alignment method known to the person of ordinary skill in the art.

There is also provided according to the invention a vector comprising a nucleic acid molecule of the invention.

Optionally a vector of the invention further comprises a promoter operably linked to the nucleic acid.

Optionally the promoter is for expression of a polypeptide encoded by the nucleic acid in mammalian cells.

Optionally the promoter is for expression of a polypeptide encoded by the nucleic acid in yeast or insect cells.

Optionally the vector is a vaccine vector.

Optionally the vector is a viral vaccine vector, a bacterial vaccine vector, an RNA vaccine vector, or a DNA vaccine vector.

A nucleic acid molecule of the invention may comprise a DNA or an RNA molecule. For embodiments in which the nucleic acid molecule comprises an RNA molecule, it will be appreciated that the molecule may comprise an RNA sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with, or identical with, any of SEQ ID NOs: 2, 4, or 6, in which each ‘T’ nucleotide is replaced by ‘U’, or the complement thereof.

For example, it will be appreciated that where an RNA vaccine vector comprising a nucleic acid of the invention is provided, the nucleic acid sequence of the nucleic acid of the invention will be an RNA sequence, so may comprise for example an RNA nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with, or identical with, any of SEQ ID NOs: 2, 4, or 6 in which each ‘T’ nucleotide is replaced by ‘U’, or the complement thereof.

Viral vaccine vectors use viruses to deliver nucleic acid (for example, DNA or RNA) into human or non-human animal cells. The nucleic acid contained in the virus encodes one or more antigens that, once expressed in the infected human or non-human animal cells, elicit an immune response. Both humoral and cell-mediated immune responses can be induced by viral vaccine vectors. Viral vaccine vectors combine many of the positive qualities of nucleic acid vaccines with those of live attenuated vaccines. Like nucleic acid vaccines, viral vaccine vectors carry nucleic acid into a host cell for production of antigenic proteins that can be tailored to stimulate a range of immune responses, including antibody, T helper cell (CD4⁺ T cell), and cytotoxic T lymphocyte (CTL, CD8⁺ T cell) mediated immunity. Viral vaccine vectors, unlike nucleic acid vaccines, also have the potential to actively invade host cells and replicate, much like a live attenuated vaccine, further activating the immune system like an adjuvant. A viral vaccine vector therefore generally comprises a live attenuated virus that is genetically engineered to carry nucleic acid (for example, DNA or RNA) encoding protein antigens from an unrelated organism. Although viral vaccine vectors are generally able to produce stronger immune responses than nucleic acid vaccines, for some diseases viral vectors are used in combination with other vaccine technologies in a strategy called heterologous prime-boost. In this system, one vaccine is given as a priming step, followed by vaccination using an alternative vaccine as a booster. The heterologous prime-boost strategy aims to provide a stronger overall immune response. Viral vaccine vectors may be used as both prime and boost vaccines as part of this strategy. Viral vaccine vectors are reviewed by Ura et al., 2014 (Vaccines 2014, 2, 624-641) and Choi and Chang, 2013 (Clinical and Experimental Vaccine Research 2013; 2:97-105).

Optionally the viral vaccine vector is based on a viral delivery vector, such as a Poxvirus (for example, Modified Vaccinia Ankara (MVA), NYVAC, AVIPOX), herpesvirus (e.g. HSV, CMV, Adenovirus of any host species), Morbillivirus (e.g. measles), Alphavirus (e.g. SFV, Sendai), Flavivirus (e.g. Yellow Fever), or Rhabdovirus (e.g. VSV)-based viral delivery vector, a bacterial delivery vector (for example, Salmonella, E. coli), an RNA expression vector, or a DNA expression vector.

Optionally the nucleic acid expression vector is a nucleic acid expression vector, and a viral pseudotype vector.

Optionally the nucleic acid expression vector is a vaccine vector.

Optionally the nucleic acid expression vector comprises, from a 5′ to 3′ direction: a promoter; a splice donor site (SD); a splice acceptor site (SA); and a terminator signal, wherein the multiple cloning site is located between the splice acceptor site and the terminator signal.

Optionally the promoter comprises a CMV immediate early 1 enhancer/promoter (CMV-IE-E/P) and/or the terminator signal comprises a terminator signal of a bovine growth hormone gene (Tbgh) that lacks a Kpnl restriction endonuclease site.

Optionally the nucleic acid expression vector further comprises an origin of replication, and nucleic acid encoding resistance to an antibiotic. Optionally the origin of replication comprises a pUC-plasmid origin of replication and/or the nucleic acid encodes resistance to kanamycin.

Optionally the vector is a pEVAC-based expression vector.

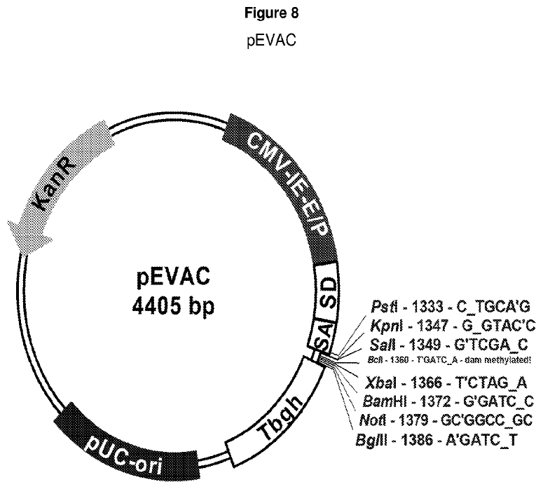

Optionally the nucleic acid expression vector comprises a nucleic acid sequence of SEQ ID NO:21 (pEVAC). The pEVAC vector has proven to be a highly versatile expression vector for generating viral pseudotypes as well as direct DNA vaccination of animals and humans. The pEVAC expression vector is described in more detail in Example 11 below. FIG. 8 shows a plasmid map for pEVAC.

There is also provided according to the invention an isolated cell comprising or transfected with a vector of the invention.

There is also provided according to the invention a fusion protein comprising a polypeptide of the invention.

There is also provided according to the invention a pseudotyped virus comprising a polypeptide of the invention.

According to the invention there is also provided a pharmaceutical composition comprising a polypeptide of the invention, and a pharmaceutically acceptable carrier, excipient, or diluent.

A pharmaceutical composition of the invention may include polypeptides of the invention in any suitable combination (for example, H5 and/or M2 and/or neuraminidase embodiments of the invention).

Optionally a pharmaceutical composition of the invention comprises:
i) a polypeptide which comprises an amino acid sequence of SEQ ID NO:7 or 8, or a polypeptide which comprises an amino acid sequence of SEQ ID NO:10 or 11, or a polypeptide which comprises an amino acid sequence of SEQ ID NO:1 or 3 (examples of H5 embodiments); and/or
ii) a polypeptide which comprises an amino acid sequence of SEQ ID NO:14 (examples of M2 embodiments); and/or
iii) a polypeptide which comprises an amino acid sequence of SEQ ID NO:16, a polypeptide which comprises an amino acid sequence of SEQ ID NO:18 (examples of neuraminidase embodiments).

According to the invention there is also provided a pharmaceutical composition comprising a nucleic acid of the invention, and a pharmaceutically acceptable carrier, excipient, or diluent.

A pharmaceutical composition of the invention may include nucleic acid molecules of the invention in any suitable combination (for example, H5 and/or M2 and/or neuraminidase embodiments of the invention).

Optionally a pharmaceutical composition of the invention comprises:
i) a nucleic acid molecule encoding a polypeptide which comprises an amino acid sequence of SEQ ID NO:7 or 8, or a nucleic acid molecule encoding a polypeptide which comprises an amino acid sequence of SEQ ID NO:10 or 11, or a nucleic acid molecule encoding a polypeptide which comprises an amino acid sequence of SEQ ID NO:1 or 3 (examples of H5 embodiments); and/or
ii) a nucleic acid molecule encoding a polypeptide which comprises an amino acid sequence of SEQ ID NO:14 (examples of M2 embodiments); and/or
iii) a nucleic acid molecule encoding a polypeptide which comprises an amino acid sequence of SEQ ID NO:16, or a nucleic acid molecule encoding a polypeptide which comprises an amino acid sequence of SEQ ID NO:18 (examples of neuraminidase embodiments).

According to the invention there is also provided a pharmaceutical composition comprising a vector of the invention, and a pharmaceutically acceptable carrier, excipient, or diluent.

Optionally a pharmaceutical composition of the invention further comprises an adjuvant for enhancing an immune response in a subject to the polypeptide, or to a polypeptide encoded by the nucleic acid, of the composition.

There is also provided according to the invention a method of inducing an immune response to an influenza virus in a subject, which comprises administering to the subject an effective amount of a polypeptide of the invention, a nucleic acid of the invention, a vector of the invention, or a pharmaceutical composition of the invention.

There is also provided according to the invention a method of immunising a subject against an influenza virus, which comprises administering to the subject an effective amount of a polypeptide of the invention, a nucleic acid of the invention, a vector of the invention, or a pharmaceutical composition of the invention.

There is further provided according to the invention a polypeptide of the invention, a nucleic acid of the invention, a vector of the invention, or a pharmaceutical composition of the invention, for use as a medicament.

There is further provided according to the invention a polypeptide of the invention, a nucleic acid of the invention, a vector of the invention, or a pharmaceutical composition of the invention, for use in the prevention, treatment, or amelioration of an influenza viral infection.

There is also provided according to the invention use of a polypeptide of the invention, a nucleic acid of the invention, a vector of the invention, or a pharmaceutical composition of the invention, in the manufacture of a medicament for the prevention, treatment, or amelioration of an influenza viral infection.

Any suitable route of administration may be used. Methods of administration include, but are not limited to, intradermal, intramuscular, intraperitoneal, parenteral, intravenous, subcutaneous, vaginal, rectal, intranasal, inhalation or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local.

Compositions may be administered in any suitable manner, such as with pharmaceutically acceptable carriers. Pharmaceutically acceptable carriers are determined in part by the particular composition being administered, as well as by the particular method used to administer the composition. Preparations for parenteral administration include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Administration can be accomplished by single or multiple doses. The dose administered to a subject in the context of the present disclosure should be sufficient to induce a beneficial therapeutic response in a subject over time, or to inhibit or prevent infection. The dose required will vary from subject to subject depending on the species, age, weight and general condition of the subject, the severity of the infection being treated, the particular composition being used and its mode of administration. An appropriate dose can be determined by one of ordinary skill in the art using only routine experimentation.

Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate. Any of the common pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used with the compositions and methods provided herein are normal saline and sesame oil.

In some embodiments, the compositions comprise a pharmaceutically acceptable carrier and/or an adjuvant. For example, the adjuvant can be alum, Freund's complete adjuvant, a biological adjuvant or immunostimulatory oligonucleotides (such as CpG oligonucleotides).

The pharmaceutically acceptable carriers (vehicles) useful in this disclosure are conventional. Remington's Pharmaceutical Sciences, by E. W. Martin, Mack Publishing Co., Easton, Pa., 15^{th} Edition (1975), describes compositions and formulations suitable for pharmaceutical delivery of one or more therapeutic compositions, such as one or more influenza vaccines, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Optionally a composition of the invention is administered intramuscularly.

Optionally the composition is administered intramuscularly, intradermally, subcutaneously by needle or by gene gun, or electroporation.

Embodiments of the invention are now described, by way of example only, with reference to the accompanying drawings, in which:

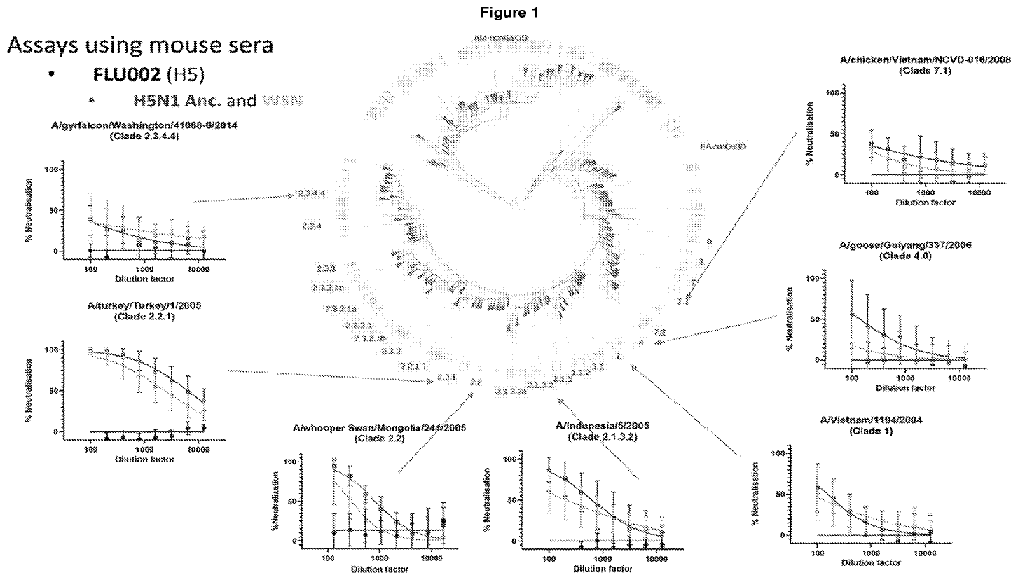

FIG. 1 shows the results of a neutralisation assay illustrating the strength of neutralising antibody responses to various pseudotyped viruses with H5 from different clades and sub-clades;

FIG. 2 shows an amino acid sequence comparison of different embodiments of polypeptides of the invention;

FIG. 3 shows an amino acid sequence comparison of different embodiments of polypeptides of the invention and prior art COBRA sequences;

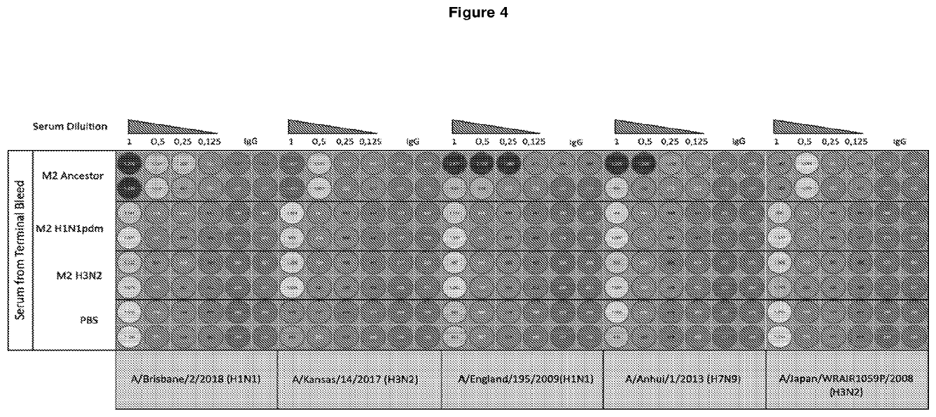

FIG. 4 shows the results of a flow cytometry-based immunofluorescence assay to test the ability of mouse sera, obtained following immunisation of mice with an embodiment of the invention, to target M2 molecules from various influenza A isolates;

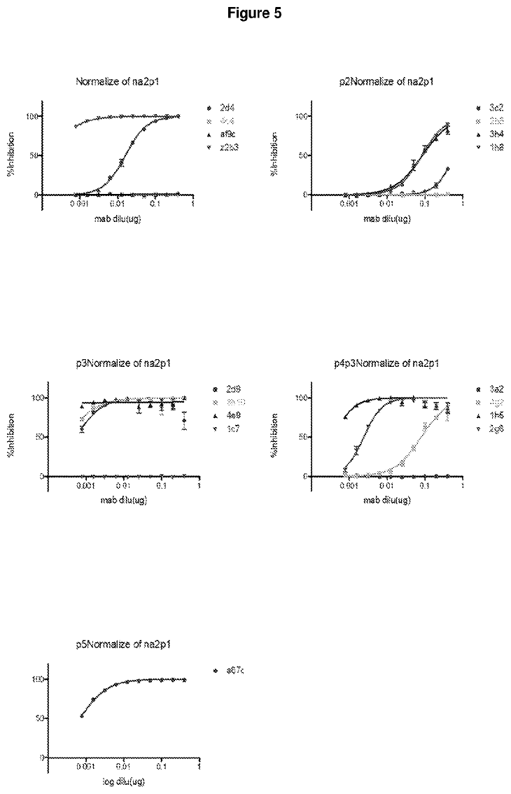

FIG. 5 shows the results of a Pseudotype-based Enzyme-Linked Lectin Assay (pELLA) using FLU_T2_NA_3;

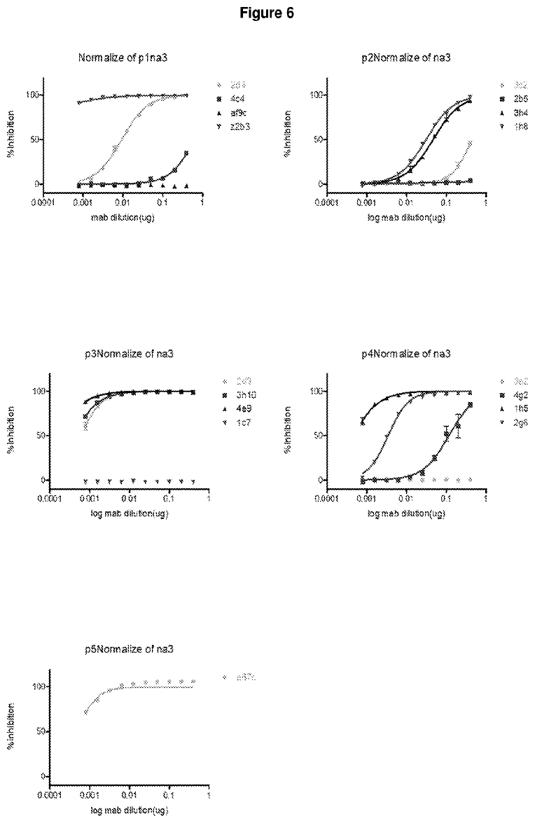

FIG. 6 shows the results of a pELLA using FLU_T2_NA_4;

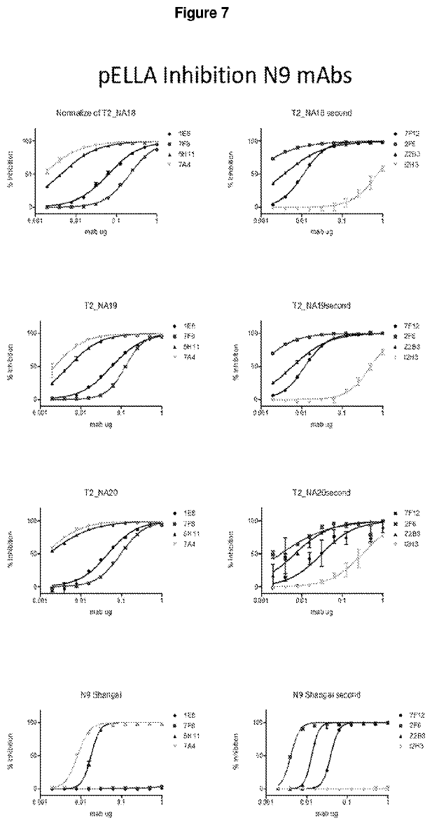

FIG. 7 shows the results of a pELLA with N9 mAbs; and

FIG. 8 shows a plasmid map for pEVAC vector.

### EXAMPLE 1—FLU_T2_HA_1

This example provides amino acid sequences of the influenza haemagglutinin H5 head and stem regions for an embodiment of the invention known as FLU_T2_HA_1. In SEQ ID NO:1 below, the amino acid residues of the stem region are shown underlined. The amino acid residues of the head region are the remaining residues.

FLU_T2_HA_1-HA0 amino acid sequence (SEQ ID NO: 1):
MEKIVLLLAIVSLVKSDQICIGYHANNSTEQVDTIMEKNVTVTHAQDILEKTHNGKLCDLDGV
KPLILRDCSVAGWLLGNPMCDEFINVPEWSYIVEKANPANDLCYPGNFNDYEELKHLLSRI
NHFEKIQIIPKSSWSDHEASSGVSSACPYQGRSSFFRNVVWLIKKNNAYPTIKRSYNNTNQ
EDLLVLWGIHHPNDAAEQTRLYQNPTTYISVGTSTLNQRLVPKIATRSKVNGQSGRMEFF
WTILKPNDAINFESNGNFIAPEYAYKIVKKGDSAIMKSELEYGNCNTKCQTPMGAINSSMPF
HNIHPLTIGECPKYVKSNRLVLATGLRNSPQRERRRKKRGLFGAIAGFIEGGWQGMVDGW
YGYHHSNEQGSGYAADKESTQKAIDGVTNKVNSIIDKMNTQFEAVGREFNNLERRIENLN
KKMEDGFLDVWTYNAELLVLMENERTLDFHDSNVKNLYDKVRLQLRDNAKELGNGCFEF
YHKCDNECMESVRNGTYDYPQYSEEARLKREEISGVKLESIGTYQILSIYSTVASSLALAIM
VAGLSLWMCSNGSLQCRICI
FLU_T2_HA_1-HA0 nucleic acid sequence (SEQ ID NO: 2):
atggaaaagattgtgctgctgctggccatcgtgtccctggtcaagagcgatcaaatctgcatcggctaccacgccaacaacag
caccgaacaggtggacaccattatggaaaagaacgtgaccgtgacacacgcccaggacatcctggaaaagaccccacaac
ggcaagctgtgcgacctggatggcgtgaagcctctgatcctgagagattgctctgtggccggctggctgctgggcaatcctatgt
gcgacgagttcatcaacgtgcccgagtggtcctatatcgtggaaaaggccaatcctgccaacgacctgtgctaccccggcaa
cttcaacgactacgaggaactgaaacatctgctgagccggatcaaccacttcgagaagatccagatcatccccaagtcctctt
ggagcgatcacgaggcctctagcggagtgtctagcgcctgtccttaccaaggcagaagcagcttcttccggaacgtcgtgtgg
ctgatcaagaagaacaacgcttaccccaccatcaagcggagctacaacaacaccaatcaagaggacctgctggtgctgtgg
ggcatccaccatcctaatgatgccgccgagcagacccggctgtaccagaatcctacaacctacatcagcgtgggcaccagc
acactgaaccagagactggtgcctaagatcgccaccagatccaaagtgaacggccagagcggccggatggaattcttctgg
accatcctgaagcctaacgacgccatcaacttcgagagcaacggcaactttatcgcccctgagtacgcctacaagatcgtga
agaagggcgacagcgccatcatgaagtccgagctggaatacggcaactgcaacaccaagtgtcagacccctatgggcgcc
atcaatagcagcatgcccttccacaacattcaccctctgaccatcggcgagtgccccaaatacgtgaagtccaacagactggt
cctggccaccgacctgagaaattctccacagagagagcggcgcagaaaaaagagaggcctgtttggaaccattaccggctt
tatcgaaggcggctggcaaggcatggttgacggatggtacggctatcaccacagcaatgagcaaggctctggctacgccgc
cgacaaagagagcacacagaaagccatcgacggcgtaaccaacaaagtgaatagcatcatcgacaagatgaacaccca
gttcgaggccgtgggcagagagttcaacaacctggaaagacggatcgagaacctgaacaagaagatggaggacggcttc
ctggacgtgtggacctataatgccgagctgctggtcctgatggaaaacgagagaaccctggacttccacgacagcaacgtga
agaacctgtacgacaaagtgcggctccagctgcgggacaatgccaaagaactcggcaacggctgcttcgagttctaccaca
agtgcgacaacgagtgcatggaaagcgtgcggaacggcacctacgactaccctcagtactctgaggaagcccggctgaag
agagaagagatcagcggagtgaagctggaatccatcggcacataccagatcctgagcatctacagcaccgtggcctcttctct
ggccctggctattatggtggctggcctgagcctgtggatgtgctctaatggcagcctccagtgccggatctgcatc
FLU_T2_HA_1-head region amino acid sequence (SEQ ID NO: 3):
THNGKLCDLDGVKPLILRDCSVAGWLLGNPMCDEFINVPEWSYIVEKANPANDLCYPGNF
NDYEELKHLLSRINHFEKIQIIPKSSWSDHEASSGVSSACPYQGRSSFFRNVVWLIKKNNA
YPTIKRSYNNTNQEDLLVLWGIHHPNDAAEQTRLYQNPTTYISVGTSTLNQRLVPKIATRSK
VNGQSGRMEFFWTILKPNDAINFESNGNFIAPEYAYKIVKKGDSAIMKSELEYGNCNTKCQ
TPMGAINSSMPFHNIHPLTIGECP

The amino acid residues at positions 156, 157, 171, 172, and 205 are shown underlined in the above sequence (and are R, S, N, A, and R, respectively).

FLU_T2_HA_1-head region nucleic acid sequence
(SEQ ID NO: 4):
acccacaacggcaagctgtgcgacctggatggcgtgaagcctctgatcc
tgagagattgctctgtggccggctggctgctgggcaatcctatgtgcga
cgagttcatcaacgtgcccgagtggtcctatatcgtggaaaaggccaat
cctgccaacgacctgtgctaccccggcaacttcaacgactacgaggaac
tgaaacatctgctgagccggatcaaccacttcgagaagatccagatcat
ccccaagtcctcttggagcgatcacgaggcctctagcggagtgtctagc
gcctgtccttaccaaggcagaagcagcttcttccggaacgtcgtgtggc
tgatcaagaagaacaacgcttaccccaccatcaagcggagctacaacaa
caccaatcaagaggacctgctggtgctgtggggcatccaccatcctaat
gatgccgccgagcagacccggctgtaccagaatcctacaacctacatca
gcgtgggcaccagcacactgaaccagagactggtgcctaagatcgccac
cagatccaaagtgaacggccagagcggccggatggaattcttctggacc
atcctgaagcctaacgacgccatcaacttcgagagcaacggcaacttta
tcgcccctgagtacgcctacaagatcgtgaagaagggcgacagcgccat
catgaagtccgagctggaatacggcaactgcaacaccaagtgtcagacc
cctatgggcgccatcaatagcagcatgcccttccacaacattcaccctc
tgaccatcggcgagtgcccc
FLU_T2_HA_1-stem region amino acid sequence (SEQ
ID NO: 5):
MEKIVLLLAIVSLVKSDQICIGYHANNSTEQVDTIMEKNVTVTHAQDIL
EKKYVKSNRLVLATGLRNSPQRERRRKKRGLFGAIAGFIEGGWQGMVDG
WYGYHHSNEQGSGYAADKESTQKAIDGVTNKVNSIIDKMNTQFEAVGRE
FNNLERRIENLNKKMEDGFLDVWTYNAELLVLMENERTLDFHDSNVKNL
YDKVRLQLRDNAKELGNGCFEFYHKCDNECMESVRNGTYDYPQYSEEAR
LKREEISGVKLESIGTYQILSIYSTVASSLALAIMVAGLSLWMCSNGSL
QCRICI

The amino acid residues at positions 416 and 434 (or at positions 148 and 166 if counting from the beginning of the stem region) are shown underlined in the above sequence (and are F and F, respectively).

FLU_T2_HA_1-stem region nucleic acid sequence
(SEQ ID NO: 6):
atggaaaagattgtgctgctgctggccatcgtgtccctggtcaagagcg
atcaaatctgcatcggctaccacgccaacaacagcaccgaacaggtgga
caccattatggaaaagaacgtgaccgtgacacacgcccaggacatcctg
gaaaagaaatacgtgaagtccaacagactggtcctggccaccggcctga
gaaattctccacagagagagcggcgcagaaagaagagaggcctgtttgg
agccattgccggctttatcgaaggcggctggcaaggcatggttgacgga
tggtacggctatcaccacagcaatgagcaaggctctggctacgccgccg
acaaagagagcacacagaaagccatcgacggcgtgaccaacaaagtgaa
tagcatcatcgacaagatgaacacccagttcgaggccgtgggcagagag
ttcaacaacctggaaagacggatcgagaacctgaacaagaagatggagg
acggcttcctggacgtgtggacctataatgccgagctgctggtcctgat
ggaaaacgagagaaccctggacttccacgacagcaacgtgaagaacctg
tacgacaaagtgcggctccagctgcgggacaatgccaaagaactcggca
acggctgcttcgagttctaccacaagtgcgacaacgagtgcatggaaag
cgtgcggaacggcacctacgactaccctcagtactctgaggaagcccgg
ctgaagagagaagagatcagcggagtgaagctggaatccatcggcacat
accagatcctgagcatctacagcaccgtggcctcttctctggccctggc
tattatggtggctggcctgagcctgtggatgtgctctaatggcagcctc
cagtgccggatctgcatc

### EXAMPLE 2

FLU_T2_HA_1 was tested for its ability to elicit a broadly neutralising antibody response to pseudotyped viruses with H5 from different clades and sub-clades.

Immunisation of Mice with DNA Vaccine:

Female BALB/c mice, 8-10 weeks old, were immunised 4 times (week 0, week 2, week 4, week 6) and bled 6-7 times (week 0, week 2, week 4, week 6, week 8, week 10, week 12) with:
50 μg FLU_T2_HA_1 DNA in pEVAC vector (see ‘H5N1 Anc.’ in FIG. 1);
50 μg A/whooper swan/Mongolia/244/2005 (H5) DNA in pEVAC vector (see ‘WSN’ in FIG. 1), which is a primary isolate strain sequenced in 2005 from a whooper swan (i.e. an H5 control); or
50 μl PBS.

DNA was injected subcutaneously into the rear flank of the mice. The DNA and the PBS are endotoxin free.

Ability of Mouse Sera to Neutralise Pseudotyped Viruses with H5 from Different Clades and Sub-Clades:

Mouse sera collected following the immunisations was tested against the following pseudotyped viruses (with H5 from different clades and sub-clades):
A/gyrfalcon/Washington/41088-6/2014 (H5, clade 2.3.4.4);
A/turkey/Turkey/1/2005 (H5, clade 2.2.1);
A/whooper Swan/Mongolia/244/2005 (H5, clade 2.2)—homologous to the H5 control;
A/Indonesia/5/2005 (H5, clade 2.1.3.2);
A/Vietnam/1194/2004 (H5, clade 1);
A/goose/Guiyang/337/2006 (H5, clade 4);
A/chicken/Vietnam/NCVD-016/2008 (H5, clade 7.1)

FIG. 1 shows the results of a neutralisation assay illustrating the strength of neutralising antibody responses to the various pseudotyped viruses. The results illustrate the ability of each vaccine to elicit broadly neutralising antibody responses to a diverse panel of pseudotyped viruses with H5 from different clades and sub-clades.

The results show that administering mice the FLU_T2_HA_1 DNA vaccine gave a significantly greater cross-clade immune response than immunisation with the A/whooper swan/Mongolia/244/2005 H5 control vaccine, and the naïve mouse serum.

### EXAMPLE 3—DESIGN OF FLU_T3_HA_1 AND FLU_T3_HA_2

This example describes the design of amino acid sequences of two further embodiments of the invention, FLU_T3_HA_1 and FLU_T3_HA_2.

As described in Example 2 above, mouse sera obtained following immunisation with FLU_T2_HA_1 DNA vaccine neutralised many clades of H5 but was less effective against clades 2.3.4 and 7.1. These two clades are currently in circulation in birds, and are among the most dominant co-circulating H5N1 viruses in poultry in Asia, with sporadic cases of infection occurring regularly in humans and other mammals.

Epitope regions in the H5 head region important for neutralisation of clade 2.3.4 and clade 7.1 were identified using available protein structural data. The amino acid sequences of these epitopes were compared with FLU_T2_HA_1 to identify amino acid positions that may have abrogated the neutralisation of these two clades by the mouse sera.

Amino acid positions within FLU_T2_HA_1 were identified that, when changed to particular amino acid residues, can elicit an antibody response that is able to neutralise clades 2.3.4 and 7.1 without abrogating the neutralisation of other clades. These positions are at amino acid residues 157, 171, 172, and 205 of the H5 protein (see positions A, B and C in FIG. 2). The influence of these mutations on the stability of the HA protein, as well as its interaction with known antibodies against clade 2.3.4 and clade 7, were checked by energetics calculations. The mutations that stabilised the protein and its interaction with such antibodies, while minimally altering the neutralisation of other clades, were selected for. The resulting new HA sequences are termed FLU_T3_HA_1 and FLU_T3_HA_2.

FIG. 2 shows an amino acid sequence comparison of FLU_T2_HA_1 with FLU_T3_HA_1 and FLU_T3_HA_2. FLU_T3_HA_1 is described in more detail in Example 4, and FLU_T3_HA_2 is described in more detail in Example 5, below.

### EXAMPLE 4—FLU_T3_HA_1

This example provides amino acid sequences of the influenza haemagglutinin H5 head and stem regions for an embodiment of the invention known as FLU_T3_HA_1. In SEQ ID NO:7 below, the amino acid residues of the stem region are shown underlined. The amino acid residues of the head region are the remaining residues.

FLU_T3_HA_1-HA0 amino acid sequence (SEQ ID NO: 7):
MEKIVLLLAIVSLVKSDQICIGYHANNSTEQVDTIMEKNVTVTHAQDILEKTHNGKLCDLDGV
KPLILRDCSVAGWLLGNPMCDEFINVPEWSYIVEKANPANDLCYPGNFNDYEELKHLLSRI
NHFEKIQIIPKSSWSDHEASSGVSSACPYQGRPSFFRNVVWLIKKNDTYPTIKRSYNNTNQ
EDLLVLWGIHHPNDAAEQTKLYQNPTTYISVGTSTLNQRLVPKIATRSKVNGQSGRMEFF
WTILKPNDAINFESNGNFIAPEYAYKIVKKGDSAIMKSELEYGNCNTKCQTPMGAINSSMPF
HNIHPLTIGECPKYVKSNRLVLATGLRNSPQRERRRKKRGLFGAIAGFIEGGWQGMVDGW
YGYHHSNEQGSGYAADKESTQKAIDGVTNKVNSIIDKMNTQFEAVGREFNNLERRIENLN
KKMEDGFLDVWTYNAELLVLMENERTLDFHDSNVKNLYDKVRLQLRDNAKELGNGCFEF
YHKCDNECMESVRNGTYDYPQYSEEARLKREEISGVKLESIGTYQILSIYSTVASSLALAIM
VAGLSLWMCSNGSLQCRICI
FLU_T3_HA_1-head region amino acid sequence (SEQ ID NO: 8):
THNGKLCDLDGVKPLILRDCSVAGWLLGNPMCDEFINVPEWSYIVEKANPANDLCYPGNF
NDYEELKHLLSRINHFEKIQIIPKSSWSDHEASSGVSSACPYQGRPSFFRNVVWLIKKNDT
YPTIKRSYNNTNQEDLLVLWGIHHPNDAAEQTKLYQNPTTYISVGTSTLNQRLVPKIATRSK
VNGQSGRMEFFWTILKPNDAINFESNGNFIAPEYAYKIVKKGDSAIMKSELEYGNCNTKCQ
TPMGAINSSMPFHNIHPLTIGECP

The amino acid residues at positions 156, 157, 171, 172, and 205 are shown underlined in the above sequence (and are R, P, D, T, and K, respectively).

FLU_T3_HA_1-stem region amino acid sequence (SEQ ID NO: 9):
MEKIVLLLAIVSLVKSDQICIGYHANNSTEQVDTIMEKNVTVTHAQDILEKKYVKSNRLVLAT
GLRNSPQRERRRKKRGLFGAIAGFIEGGWQGMVDGWYGYHHSNEQGSGYAADKESTQ
KAIDGVTNKVNSIIDKMNTQFEAVGREFNNLERRIENLNKKMEDGFLDVWTYNAELLVLME
NERTLDEHDSNVKNLYDKVRLQLRDNAKELGNGCFEFYHKCDNECMESVRNGTYDYPQY
SEEARLKREEISGVKLESIGTYQILSIYSTVASSLALAIMVAGLSLWMCSNGSLQCRICI

The amino acid residues at positions 416 and 434 are shown underlined in the above sequence (and are F and F, respectively).

### EXAMPLE 5—INFLUENZA H5 T3_HA_2

This example provides amino acid sequences of the influenza H5 head and stem regions for an embodiment of the invention known as FLU_T3_HA_2. In SEQ ID NO:4 below, the amino acid residues of the stem region are shown underlined. The amino acid residues of the head region are the remaining residues.

FLU_T3_HA_2-HA0 amino acid sequence (SEQ ID NO: 10):
MEKIVLLLAIVSLVKSDQICIGYHANNSTEQVDTIMEKNVTVTHAQDILEKTHNGKLCDLDGV
KPLILRDCSVAGWLLGNPMCDEFINVPEWSYIVEKANPANDLCYPGNFNDYEELKHLLSRI
NHFEKIQIIPKSSWSDHEASSGVSSACPYQGRPSFFRNVVWLIKKNNTYPTIKRSYNNTNQ
EDLLVLWGIHHPNDAAEQTKLYQNPTTYISVGTSTLNQRLVPKIATRSKVNGQSGRMEFF
WTILKPNDAINFESNGNFIAPEYAYKIVKKGDSAIMKSELEYGNCNTKCQTPMGAINSSMPF
HNIHPLTIGECPKYVKSNRLVLATGLRNSPQRERRRKKRGLFGAIAGFIEGGWQGMVDGW
YGYHHSNEQGSGYAADKESTQKAIDGVTNKVNSIIDKMNTQFEAVGREFNNLERRIENLN
KKMEDGFLDVWTYNAELLVLMENERTLDFHDSNVKNLYDKVRLQLRDNAKELGNGCFEF
YHKCDNECMESVRNGTYDYPQYSEEARLKREEISGVKLESIGTYQILSIYSTVASSLALAIM
VAGLSLWMCSNGSLQCRICI
FLU_T3_HA_2-head region amino acid sequence (SEQ ID NO: 11):
THNGKLCDLDGVKPLILRDCSVAGWLLGNPMCDEFINVPEWSYIVEKANPANDLCYPGNF
NDYEELKHLLSRINHFEKIQIIPKSSWSDHEASSGVSSACPYQGRPSFFRNVVWLIKKNNT
YPTIKRSYNNTNQEDLLVLWGIHHPNDAAEQTKLYQNPTTYISVGTSTLNQRLVPKIATRSK
VNGQSGRMEFFWTILKPNDAINFESNGNFIAPEYAYKIVKKGDSAIMKSELEYGNCNTKCQ
TPMGAINSSMPFHNIHPLTIGECP

The amino acid residues at positions 156, 157, 171, 172, and 205 are shown underlined in the above sequence (and are R, P, N, T, and K, respectively).

FLU_T3_HA_2-stem region amino acid sequence (SEQ ID NO: 12)
MEKIVLLLAIVSLVKSDQICIGYHANNSTEQVDTIMEKNVTVTHAQDILEKKYVKSNRLVLAT
GLRNSPQRERRRKKRGLFGAIAGFIEGGWQGMVDGWYGYHHSNEQGSGYAADKESTQ
KAIDGVTNKVNSIIDKMNTQFEAVGREFNNLERRIENLNKKMEDGFLDVWTYNAELLVLME
NERTLDFHDSNVKNLYDKVRLQLRDNAKELGNGCFEFYHKCDNECMESVRNGTYDYPQY
SEEARLKREEISGVKLESIGTYQILSIYSTVASSLALAIMVAGLSLWMCSNGSLQCRICI

The amino acid residues at positions 416 and 434 are shown underlined in the above sequence (and are F and F, respectively).

### EXAMPLE 6—COMPARISON OF FLU_T3_HA_1 AND FLU_T3_HA_2 WITH PRIOR ART COBRA H5 TIER 2 DESIGN

FIG. 3 shows an amino acid comparison of FLU_T3_HA_1 and FLU_T3_HA_2 with a prior art COBRA H5 Tier 2 design. There are amino acid differences at three positions (A, B, and C) in the head region which have been introduced in FLU_T3_HA_1 and FLU_T3_HA_2 to increase the affinity of the antigen towards antibodies of important clades. The amino acid differences are at residue numbers 156, 157, 171, 172, and 205 of the head region. There are additional amino acid differences at two positions (C and D) in the stem region which have been introduced in FLU_T3_HA_1 and FLU_T3_HA_2 to stabilise the stem region in both the pre- and post-fusion state. The amino acid differences are at residue numbers 416 and 434 of the stem region.

### EXAMPLE 7—FLU_T2_M2_1

This example provides the amino acid and nucleic acid sequences of the influenza M2 region for an embodiment of the invention known as FLU_T2_M2_1.

FLU_T2_M2_1-amino acid sequence (SEQ ID NO: 14):
MSLLTEVETPTRNGWECRCSDSSDPLVIAASIIGILHLILWILDRLFFKCIYRRLKYGLKRGP
STEGVPESMREEYRQKQQSAVDVDDGHFVNIELE
FLU_T2_M2_1-nucleic acid sequence (SEQ ID NO: 15):
ATGTCTCTGCTGACCGAGGTGGAAACCCCTACCAGAAATGGCTGGGAGTGCAGATGC
AGCGACAGCAGCGATCCTCTGGTTATCGCCGCCAGCATCATCGGCATCCTGCACCTG
ATCCTGTGGATCCTGGACCGGCTGTTCTTCAAGTGCATCTACCGGCGGCTGAAGTAC
GGCCTGAAGAGAGGCCCTTCTACAGAGGGCGTGCCCGAGAGCATGCGGGAAGAGTA
CAGACAGAAACAGCAGAGCGCCGTGGACGTGGACGATGGCCACTTCGTGAACATCGA
GCTGGAA

### EXAMPLE 8—IMMUNE RESPONSE ELICITED BY FLU_T2_M2_1

This example describes a flow cytometry-based immunofluorescence assay to test the ability of mouse sera, obtained following immunisation of mice with FLU_T2_M2_1 DNA vaccine, to target M2 molecules from influenza A isolates of different subtypes.

Immunisation of Mice with DNA Vaccine:

4 groups of 6 Balb/c mice, 8-10 weeks old, were immunised 4 times (week 0, week 2, week 4, week 6) and bled 6 times (week 0, week 2, week 4, week 6, week 8, week 10) with:
50 μg FLU_T2_M2_1 DNA in pEVAC vector (see ‘M2 ancestor.’ in FIG. 5);
50 μg FLU_T1_M2_1 DNA in pEVAC vector (M2 from H1N1pdm, see ‘M2 H1N1’ in FIG. 5);
50 μg FLU_T1_M2_2 DNA in pEVAC vector (M2 from H3N2, see ‘M2 H3N2’ in FIG. 5); or
50 μl PBS.

DNA was injected subcutaneously into the rear flank of the mice. The DNA and the PBS are endotoxin free.

Ability of Mouse Sera to Target M2 from Influenza Isolates of Different Subtypes:

HEK293T cells were transfected with pEVAC vector expressing M2 DNA from the following isolates:
A/Brisbane/2/2018 (H1N1);
A/Kansas/14/2017 (H3N2);
A/England/195/2009 (H1N1);
A/Anhui/1/2013 (H7N9); and
A/Japan/WRAIR1059P/2008 (H3N2)

Serum was pooled for each group (six mice per group), serially diluted and incubated with cells for 30 minutes at room temperature. Mouse IgG isotype antibody was used as negative control staining. After incubation, cells were washed twice in PBS, and then incubated with Goat anti-mouse AF647 secondary antibody for 30 minutes at room temperature, in the dark. Before FACS analysis, cells were washed with PBS another two times. Analysis was performed using Attune NxT FACS (Thermo Fisher).

FIG. 5 shows the results of a flow cytometry-based immunofluorescence assay illustrating the ability of the mouse serum antibodies to target M2s from the different influenza isolates. The results illustrate the ability of each vaccine to target M2 from influenza isolates of different subtypes.

The results show that administering mice the FLU_T2_M2_1 DNA vaccine (M2 ancestor) elicited a significantly greater immune response against M2 across different influenza subtypes than immunisation with M2 from H1N1 or H3N2 isolates, and the naïve mouse serum.

### EXAMPLE 9—FLU_T2_NA_3 AND FLU_T2_NA_4

This example provides the amino acid and nucleic acid sequences of the influenza neuraminidase region for embodiments of the invention known as FLU_T2_NA_3 and FLU_T2_NA_4.

FLU_T2_NA_3 (N1_FINAL_2)-amino acid sequence (SEQ ID NO: 16):
MNPNQKIITIGSICMVVGIISLILQIGNIISIWVSHSIQTGNQNQPETCNQSIITYENNTWVNQT
YVNISNTNFVAEQAVASVALAGNSSLCPISGWAIYSKDNGIRIGSKGDVFVIREPFISCSHLE
CRTFFLTQGALLNDKHSNGTVKDRSPYRTLMSCPVGEAPSPYNSRFESVAWSASACHDG
ISWLTIGISGPDNGAVAVLKYNGIITDTIKSWRNNILRTQESECACINGSCFTIMTDGPSNGQ
ASYKIFKIEKGKVVKSVELNAPNYHYEECSCYPDAGEVMCVCRDNWHGSNRPWVSFNQN
LEYQIGYICSGVFGDNPRPNDGTGSCGPVSSNGAYGVKGFSFKYGKGVWIGRTKSTSSR
SGFEMIWDPNGWTETDSSFSVKQDIVAITDWSGYSGSFVQHPELTGLDCMRPCFWVELI
RGRPKENTIWTSGSSISFCGVNSDTVGWSWPDGAELPFTIDK
FLU_T2_NA_3 (N1_FINAL_2)-nucleic acid sequence (SEQ ID NO: 17):
atgaatccaaatcagaaaataataaccattgggtcaatctgtatggtagttggaataatcagcctaatattacaaattgggaaca
taatctcaatatgggttagccattcaattcagactggaaatcaaaaccaacctgaaacatgcaaccaaagcatcattacttatga
aaacaacacttgggtgaatcaaacatatgttaacatcagcaataccaattttgttgctgaacaggctgtagcttcagtggcattag
cgggcaattcctctctctgccccattagtgggtgggctatatacagcaaggacaatggcataaggattggttccaagggagatgt
atttgtcataagagagccattcatttcatgctcccacttggaatgcaggaccttttttctgactcaaggagccttgttgaatgacaaa
cattccaatggaaccgttaaagacagaagcccctacagaaccttaatgagctgtcctgttggtgaggctccctctccatacaatt
caaggtttgagtcggttgcttggtcagcaagtgcttgccatgatggcattagctggttgacaattggaatttccgggccagacaat
ggggcagtggctgtattgaaatacaatggcataataacagacactatcaaaagttggagaaacaacatattgaggacacaa
gagtctgaatgtgcctgcataaatggttcttgctttactataatgaccgatggaccaagtaatgggcaggcctcatacaagattttc
aagatagagaaggggaaggtagtcaaatcagtcgagttgaatgcccctaattaccactacgaggaatgttcctgttatcctgat
gctggcgaagtaatgtgtgtgtgcagggataattggcatggttcgaatcgaccatgggtgtctttcaatcaaaatctggagtatca
aataggatacatatgcagtggggttttcggagacaatccacgccccaatgatggaacaggcagctgtggtccagtgtcttctaat
ggagcatatggagtaaagggattttcatttaagtacggcaagggtgtttggatagggagaactaagagcactagttccaggagt
ggatttgagatgatttgggatcccaatggatggacagagacagatagtagtttctcagtgaagcaagatattgtagcaataactg
attggtcaggatatagcgggagttttgtccaacatccagaattaacagggctggactgcatgaggccttgcttctgggttgaacta
atcagaggacggcctaaggagaacacaatctggactagtgggagcagcatttccttctgtggtgtaaatagcgacactgtggg
ttggtcttggccagacggtgctgagttgccattcaccattgacaag
FLU_T2_NA_4 (N1_FINAL_3)-amino acid sequence (SEQ ID NO: 18):
MNPNQKIITIGSICMVVGIISLILQIGNIISIWVSHSIQTGNQNHPETCNQSIITYENNTWVNQT
YVNISNTNVVAGQDATSVILAGNSSLCPISGWAIYSKDNGIRIGSKGDVFVIREPFISCSHLE
CRTFFLTQGALLNDKHSNGTVKDRSPYRTLMSCPVGEAPSPYNSRFESVAWSASACHDG
MGWLTIGISGPDNGAVAVLKYNGIITDTIKSWRNNILRTQESECACVNGSCFTIMTDGPSN
GQASYKIFKIEKGKVIKSIELNAPNYHYEECSCYPDTGKVMCVCRDNWHGSNRPWVSFDQ
NLDYQIGYICSGVFGDNPRPNDGTGSCGPVSSNGANGVKGFSFRYGNGVWIGRTKSTSS
RSGFEMIWDPNGWTETDSSFSVKQDIVAITDWSGYSGSFVQHPELTGLDCMRPCFWVEL
IRGQPKENTIWTSGSSISFCGVNSDTVGWSWPDGAELPFTIDK
FLU_T2_NA_4 (N1_FINAL_3) nucleic acid sequence (SEQ ID NO: 19):
atgaatccaaatcaaaaaataataaccattgggtcaatctgtatggtagttggaataattagcctaatattgcaaatagggaatat
aatctcaatatgggttagccattcaattcaaactggaaatcaaaaccatcctgaaacatgcaaccaaagcatcattacctatga
aaataacacctgggtgaatcaaacatatgttaacattagcaatactaacgttgttgctggacaggatgcaacttcagtgatattag
ccggcaattcctctctttgccccatcagtgggtgggctatatacagcaaagacaatggcataagaattggttccaaaggagacg
tttttgtcataagagagccatttatttcatgctctcacttggaatgcaggaccttttttctgactcaaggcgccttgctgaatgacaagc
attcaaatgggaccgtcaaggacagaagcccctatagaaccttaatgagctgccctgttggtgaagctccgtctccgtacaattc
aaggttcgaatcggttgcttggtcagcaagtgcatgccatgatggcatgggctggctaacaatcggaatttccggtccagataat
ggagcagtggctgtattaaaatacaatggtataataacagacaccatcaaaagttggaggaacaacatattgagaacgcaa
gagtctgaatgtgcctgtgtaaatggttcatgttttactataatgaccgatggcccaagtaatgggcaggcctcgtacaaaattttc
aagatagagaaggggaaggttattaaatcaattgagttgaatgcacctaattaccactacgaggaatgttcctgttaccctgata
caggtaaagtgatgtgtgtgtgcagagacaattggcatggttcgaatcgaccatgggtgtctttcgatcaaaatctggattatcaa
ataggatacatctgcagtggggttttcggtgacaatccgcgtcccaatgatggaacaggcagctgtggtccagtgtcttctaatgg
agcaaatggagtaaagggattttcatttaggtatggtaatggtgtttggataggaagaactaaaagtaccagttccagaagcgg
gtttgagatgatttgggatcctaatggatggacagagactgatagtagtttctctgtgaaacaagatattgtagcaataactgattg
gtcagggtacagcgggagtttcgttcaacatcctgagctaacagggctggactgcatgaggccttgcttctgggttgaattaatca
ggggacaacctaaagagaacacaatctggactagtgggagcagcatttccttttgtggcgtaaatagtgatactgtaggttggtc
ttggccagacggtgctgagttgccattcaccattgacaag

### EXAMPLE 10—ANTIBODY INHIBITION OF NEURAMINIDASE ACTIVITY OF FLU_T2_NA_3 AND FLU_T2_NA_4

This example describes screening of neuraminidase polypeptides according to embodiments of the invention (FLU_T2_NA_3 and FLU_T2_NA_4) against a panel of monoclonal antibodies that recognise different neuraminidase epitopes.

Neuraminidase vaccines elicit binding antibodies or antibodies that inhibit the activity of the neuraminidase enzyme. This has been shown to correlate with reduction of severity of disease, but not necessarily protection from infection. They also reduce transmission from infected vaccinated people, as the viruses require the NA activity to exit from infected cells.

Pseudotype Based Enzyme-Linked Lectin Assay (pELLA)

Lentiviral pseudotypes are produced bearing the neuraminidase of selected influenza virus strains (e.g. the N9 from A/Shanghai/02/2013 (H7N9) or of a polypeptide according to an embodiment of the invention (e.g. T2_NA 3).

These pseudotypes bearing NA are used to digest the carbohydrate fetuin from pre-coated ELISA plates in a dilution series. The resulting product from the digested fetuin contains terminal galactose residues that can be recognised by the peanut lectin (conjugated to horseradish peroxidase).

The more the NA digests the fetuin, the more galactose is exposed, so more peanut lectin (HRPO) attaches to the galactose. An ELISA-based readout proportional to the enzymatic activity of the NA is obtained (Couzens et al., J Virol Methods. 2014 Dec. 15; 210:7-14.)

The NA-pseudotypes are first titrated, then an inhibition assay is performed with antibodies or serum to ‘knock down’ the activity of the enzyme with antibodies. As this is a functional assay, it will only detect antibodies interfering with the enzymatic activity of the NA.

### FIG. 5:

Panel of monoclonal antibodies tested against FLU_T2_NA_3 (N1_FINAL_2):
Strong inhibition of NA activity by: 2D4, Z2B3, 3H4, 1 H8, 2D9, 3H10, 4E9, 4G2, 1H5,2G6, A67C
Weak inhibition by: 3C2
No inhibition by: AF9C, 4C4, 2B5, 1C7, 3A2 FLU_T2_NA_3 (=N1_FINAL_2=na2=na2p1 in FIG. 5)

### FIG. 6:

Panel of monoclonal antibodies tested against FLU_T2_NA_4 (N1_FINAL_3):
Strong inhibition of NA activity by: Z2B3, 2D4, 1 H8, 3H4, 2D9, 3H10, 4E9, 1 H5, 2G6, 4G2, A67C
Weak inhibition by: 4C4, 3C2
No inhibition by: AF9C, 2B5, 1C7, 3A2
   FLU_T2_NA 4 (=N1 FINAL_3=p1na3=na3 in FIG. 6)

### FIG. 7:

Panel of monoclonal antibodies tested against FLU_T2_NA_18 (N9_FINAL_1), FLU_T2_NA_19 (N9_FINAL_2), FLU_T2_NA_20 (N9_FINAL_3):
Strong inhibition of NA activity by: 1E8, 7F8, 5H11, 7A4, 7F12, 2F6, Z2B3, 1E8
Weak inhibition by: 12H3
No inhibition by: N/A

For the wild type N9 (A/Shanghai/02/2013):
Strong inhibition by: 1E8, 5H11, 7A4, 2F6, 7F12, Z2B3
No inhibition by: 7F8 and 12H3

It was concluded from the results described above, and shown in FIGS. 5-7, that neuraminidase polypeptides according to embodiments of the invention (FLU_T2_NA_3 and FLU_T2_NA_4) contain epitopes conserved between N1 from seasonal H1N1, pandemic H1N1 and N1 from avian H5N1, as well as conserved epitope (Z2B3 mAb) between N1 and N9.

### Monoclonal Antibody Panel:

mAbs from Honqauan Wan, FDA:

| | | |
|---|---|---|
| mAb_1E8 | N9 | Wan et al., Journal of Virology, 2013, Vol. 87(16): |
| | | 9290-9300; |
| mAb_7F8 | N9 | Wan et al., Journal of Virology, 2018, Vol. 92(4): |
| | | 1-17; |
| mAb_11B2 | N9 | Wan et al., Nat Commun., 2015, Feb. 10; 6: 6114; |
| mAb_5H11 | N9 | |
| mAb_7A4 | N9 | |
| mAb_7F12 | N9 | |
| mAb_2F6 | N9 | |
| mAb_3A2 | N1 | |
| mAb_4G2 | N1 | |
| mAb_1H5 | N1 | |
| mAb_2G6 | N1 | |
| mAb_2D9 | N1 | |
| mAb_3H10 | N1 | |
| mAb_4E9 | N1 | |
| mAb_1C7 | N1 | |
| mAb_3C2 | N1 | |
| mAb_2B5 | N1 | |
| mAb_3H4 | N1 | |
| mAb_1H8 | N1 | |
| mAb_2D4 | N1 | |
| mAb_4C4 | N1 | |

mAbs from Alain Townsend, Oxford:

| | | |
|---|---|---|
| mAb_AF9C | N1 from seasonal and | Rijal et al., Journal of |
| | pandemic H1N1 | Virology, February 2020 |
| | | Volume 94 Issue 4, 1-17; |
| mAb_Z2B3 | N1 and N9 | Rijal et al., Journal of |
| | | Virology, February 2020 |
| | | Volume 94 Issue 4, 1-17 |

### FACS Binding Assay:

The NA is expressed on the cell surface of HEK293T/17 cells and serum/mAbs are allowed to bind to it. Binding is detected with a secondary antibody directed to the mouse or human serum antibodies. The cells are passed through a Fluorescent activated cell sampler (FACS cytometer) and the amount of binding present in a sample is measured. This binding is irrespective of whether the antibodies interfere with the enzymatic activity. These may be antibodies that act through ADCC mechanisms through immune cells.

### EXAMPLE 11—PEVAC EXPRESSION VECTOR

FIG. 8 shows a map of the pEVAC expression vector. The sequence of the multiple cloning site of the vector is given below, followed by its entire nucleotide sequence.

| Sequence of pEVAC Multiple Cloning Site (MCS) (SEQ ID NO: 20): | |
|---|---|
| 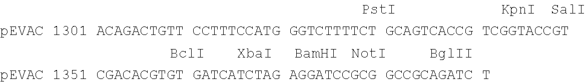 | |
| Entire Sequence of pEVAC (SEQ ID NO: 21): | |
| CMV-IE-E/P: 248-989 CMV immediate early 1 enhancer/promoter | |
| KanR: 3445-4098 Kanamycin resistance | |
| SD: 990-1220 Splice donor | |
| SA: 1221-1343 Splice acceptor | |
| Tbgh: 1392-1942 Terminator signal from bovine growth hormone | |
| pUC-ori: 2096-2769 pUC-plasmid origin of replication | |
| 1 | TCGCGCGTTT CGGTGATGAC GGTGAAAACC TCTGACACAT GCAGCTCCCG |
| 51 | GAGACGGTCA CAGCTTGTCT GTAAGCGGAT GCCGGGAGCA GACAAGGCCG |
| 101 | TCAGGGCGCG TCAGCGGGTG TTGGCGGGTG TCGGGGCTGG CTTAACTATG |
| 151 | CGGCATCAGA GCAGATTGTA CTGAGAGTGC ACCATATGCG GTGTGAAATA |
| 201 | CCGCACAGAT GCGTAAGGAG AAAATACCGC ATCAGATTGG CTATTGGCCA |
| 251 | TTGCATACGT TGTATCCATA TCATAATATG TACATTTATA TTGGCTCATG |
| 301 | TCCAACATTA CCGCCATGTT GACATTGATT ATTGACTAGT TATTAATAGT |
| 351 | AATCAATTAC GGGGTCATTA GTTCATAGCC CATATATGGA GTTCCGCGTT |
| 401 | ACATAACTTA CGGTAAATGG CCCGCCTGGC TGACCGCCCA ACGACCCCCG |
| 451 | CCCATTGACG TCAATAATGA CGTATGTTCC CATAGTAACG CCAATAGGGA |
| 501 | CTTTCCATTG ACGTCAATGG GTGGAGTATT TACGGTAAAC TGCCCACTTG |
| 551 | GCAGTACATC AAGTGTATCA TATGCCAAGT ACGCCCCCTA TTGACGTCAA |
| 601 | TGACGGTAAA TGGCCCGCCT GGCATTATGC CCAGTACATG ACCTTATGGG |
| 651 | ACTTTCCTAC TTGGCAGTAC ATCTACGTAT TAGTCATCGC TATTACCATG |
| 701 | GTGATGCGGT TTTGGCAGTA CATCAATGGG CGTGGATAGC GGTTTGACTC |
| 751 | ACGGGGATTT CCAAGTCTCC ACCCCATTGA CGTCAATGGG AGTTTGTTTT |
| 801 | GGCACCAAAA TCAACGGGAC TTTCCAAAAT GTCGTAACAA CTCCGCCCCA |
| 851 | TTGACGCAAA TGGGCGGTAG GCGTGTACGG TGGGAGGTCT ATATAAGCAG |
| 901 | AGCTCGTTTA GTGAACCGTC AGATCGCCTG GAGAGGCCAT CCACGCTGTT |
| 951 | TTGACCTCCA TAGAAGACAC CGGGACCGAT CCAGCCTCCA TCGGCTCGCA |
| 1001 | TCTCTCCTTC ACGCGCCCGC CGCCCTACCT GAGGCCGCCA TCCACGCCGG |
| 1051 | TTGAGTCGCG TTCTGCCGCC TCCCGCCTGT GGTGCCTCCT GAACTGCGTC |
| 1101 | CGCCGTCTAG GTAAGTTTAA AGCTCAGGTC GAGACCGGGC CTTTGTCCGG |
| 1151 | CGCTCCCTTG GAGCCTAGCT AGACTCAGCC GGCTCTCCAC GCTTTGCCTG |
| 1201 | ACCCTGCTTG CTCAACTCTA GTTAACGGTG GAGGGCAGTG TAGTCTGAGC |
| 1251 | AGTACTCGTT GCTGCCGCGC GCGCCACCAG ACATAATAGC TGACAGACTA |
| 1301 | ACAGACTGTT CCTTTCCATG GGTCTTTTCT GCAGTCACCG TCGGTACCGT |
| 1351 | CGACACGTGT GATCATCTAG AGGATCCGCG GCCGCAGATC TGCTGTGCCT |
| 1401 | TCTAGTTGCC AGCCATCTGT TGTTTGCCCC TCCCCCGTGC CTTCCTTGAC |
| 1451 | CCTGGAAGGT GCCACTCCCA CTGTCCTTTC CTAATAAAAT GAGGAAATTG |
| 1501 | CATCGCATTG TCTGAGTAGG TGTCATTCTA TTCTGGGGGG TGGGGTGGGG |
| 1551 | CAGGACAGCA AGGGGGAGGA TTGGGAAGAC AATAGGAGGC ATGCTGGGGA |
| 1601 | TGGGGTGGGG TCTATGGCTA CCCAGGTGCT GAAGAATTGA CCCGGTTCCT |
| 1651 | CCTGGGCCAG AAAGAAGCAG GCACATCCCC TTCTCTGTGA CACACCCTGT |
| 1701 | CCACGCCCCT GGTTCTTAGT TCCAGCCCCA CTCATAGGAC ACTCATAGCT |
| 1751 | CAGGAGGGCT CCGCCTTCAA TCCCACCCGC TAAAGTACTT GGAGCGGTCT |
| 1801 | CTCCCTCCCT CATCAGCCCA CCAAACCAAA CCTAGCCTCC AAGAGTGGGA |
| 1851 | AGAAATTAAA GCAAGATAGG CTATTAAGTG CAGAGGGAGA GAAAATGCCT |
| 1901 | CCAACATGTG AGGAAGTAAT GAGAGAAATC ATAGAATTTT AAGGCCATGA |
| 1351 | TTTAAGGCCA TCATGGCCTT AATCTTCCGC TTCCTCGCTC ACTGACTCGC |
| 2001 | TGCGCTCGGT CGTTCGGCTG CGGCGAGCGG TATCAGGTCA CTCAAAGGCG |
| 2051 | GTAATACGGT TATCCACAGA ATCAGGGGAT AACGCAGGAA AGAACATGTG |
| 2101 | AGCAAAAGGC CAGCAAAAGG CCAGGAACCG TAAAAAGGCC GCGTTGCTGG |
| 2151 | CGTTTTTCCA TAGGCTCCGC CCCCCTGACG AGCATCACAA AAATCGACGC |
| 2201 | TCAAGTCAGA GGTGGCGAAA CCCGACAGGA CTATAAAGAT ACCAGGCGTT |
| 2251 | TCCCCCTGGA AGCTCCCTCG TGCGCTCTCC TGTTCCGACC CTGCCGCTTA |
| 2301 | CCGGATACCT GTCCGCCTTT CTCCCTTCGG GAAGCGTGGC GCTTTCTCAT |
| 2351 | AGCTCACGCT GTAGGTATCT CAGTTCGGTG TAGGTCGTTC GCTCCAAGCT |
| 2401 | GGGCTGTGTG CACGAACCCC CCGTTCAGCC CGACCGCTGC GCCTTATCCG |
| 2451 | GTAACTATCG TCTTGAGTCC AACCCGGTAA GACAGGACTT ATCGCCACTG |
| 2501 | GCAGCAGCCA CTGGTAACAG GATTAGCAGA GCGAGGTATG TAGGCGGTGC |
| 2551 | TACAGAGTTC TTGAAGTGGT GGCCTAACTA CGGCTACACT AGAAGAACAG |
| 2601 | TATTTGGTAT CTGCGCTCTG CTGAAGCCAG TTACCTTCGG AAAAAGAGTT |
| 2651 | GGTAGCTCTT GATCCGGCAA ACAAACCACC GCTGGTAGCG GTGGTTTTTT |
| 2701 | TGTTTGCAAG CAGCAGATTA CGCGCAGAAA AAAAGGATCT CAAGAAGATC |
| 2751 | CTTTGATCTT TTCTACGGGG TCTGACGCTC AGTGGAACGA AAACTCACGT |
| 2801 | TAAGGGATTT TGGTCATGAG ATTATCAAAA AGGATCTTCA CCTAGATCCT |
| 2851 | TTTAAATTAA AAATGAAGTT TTAAATCAAT CTAAAGTATA TATGAGTAAA |
| 2301 | CTTGGTCTGA CAGTTACCAA TGCTTAATCA GTGAGGCACC TATCTCAGCG |
| 2351 | ATCTGTCTAT TTCGTTCATC CATAGTTGCC TGACTCGGGG GGGGGGGGCG |
| 3001 | CTGAGGTCTG CCTCGTGAAG AAGGTGTTGC TGACTCATAC CAGGCCTGAA |
| 3051 | TCGCCCCATC ATCCAGCCAG AAAGTGAGGG AGCCACGGTT GATGAGAGCT |
| 3101 | TTGTTGTAGG TGGACCAGTT GGTGATTTTG AACTTTTGCT TTGCCACGGA |
| 3151 | ACGGTCTGCG TTGTCGGGAA GATGCGTGAT CTGATCCTTC AACTCAGCAA |
| 3201 | AAGTTCGATT TATTCAACAA AGCCGCCGTC CCGTCAAGTC AGCGTAATGC |
| 3251 | TCTGCCAGTG TTACAACCAA TTAACCAATT CTGATTAGAA AAACTCATCG |
| 3301 | AGCATCAAAT GAAACTGCAA TTTATTCATA TCAGGATTAT CAATACCATA |
| 3351 | TTTTTGAAAA AGCCGTTTCT GTAATGAAGG AGAAAACTCA CCGAGGCAGT |
| 3401 | TCCATAGGAT GGCAAGATCC TGGTATCGGT CTGCGATTCC GACTCGTCCA |
| 3451 | ACATCAATAC AACCTATTAA TTTCCCCTCG TCAAAAATAA GGTTATCAAG |
| 3501 | TGAGAAATCA CCATGAGTGA CGACTGAATC CGGTGAGAAT GGCAAAAGCT |
| 3551 | TATGCATTTC TTTCCAGACT TGTTCAACAG GCCAGCCATT ACGCTCGTCA |
| 3601 | TCAAAATCAC TCGCATCAAC CAAACCGTTA TTCATTCGTG ATTGCGCCTG |
| 3651 | AGCGAGACGA AATACGCGAT CGCTGTTAAA AGGACAATTA CAAACAGGAA |
| 3701 | TCGAATGCAA CCGGCGCAGG AACACTGCCA GCGCATCAAC AATATTTTCA |
| 3751 | CCTGAATCAG GATATTCTTC TAATACCTGG AATGCTGTTT TCCCGGGGAT |
| 3801 | CGCAGTGGTG AGTAACCATG CATCATCAGG AGTACGGATA AAATGCTTGA |
| 3851 | TGGTCGGAAG AGGCATAAAT TCCGTCAGCC AGTTTAGTCT GACCATCTCA |
| 3301 | TCTGTAACAT CATTGGCAAC GCTACCTTTG CCATGTTTCA GAAACAACTC |
| 3351 | TGGCGCATCG GGCTTCCCAT ACAATCGATA GATTGTCGCA CCTGATTGCC |
| 4001 | GGACATTATC GCGAGCCCAT TTATACCCAT ATAAATCAGC ATCCATGTTG |
| 4051 | GAATTTAATC GCGGCCTCGA GCAAGACGTT TCCCGTTGAA TATGGCTCAT |
| 4101 | AACACCCCTT GTATTACTGT TTATGTAAGC AGACAGTTTT ATTGTTCATG |
| 4151 | ATGATATATT TTTATCTTGT GCAATGTAAC ATCAGAGATT TTGAGACACA |
| 4201 | ACGTGGCTTT CCCCCCCCCC CCATTATTGA AGCATTTATC AGGGTTATTG |
| 4251 | TCTCATGAGC GGATACATAT TTGAATGTAT TTAGAAAAAT AAACAAATAG |
| 4301 | GGGTTCCGCG CACATTTCCC CGAAAAGTGC CACCTGACGT CTAAGAAACC |
| 4351 | ATTATTATCA TGACATTAAC CTATAAAAAT AGGCGTATCA CGAGGCCCTT |
| 4401 | TCGTC |

1. An isolated polypeptide comprising a haemagglutinin subtype 5 (H5) globular head domain, and optionally a haemagglutinin stem domain, with the following amino acid residues at positions 156, 157, 171, 172, and 205 of the head domain:
156: R;
157: P or S, preferably P;
171: D or N;
172: T or A, preferably T; and
205: K or R, preferably K

2. An isolated polypeptide according to claim 1, with the following amino acid residues at positions 156, 157, 171, 172, and 205 of the head domain:
156: R;
157: P;
171: D;
172: T; and
205: K

3. An isolated polypeptide according to claim 1 or 2, which comprises an amino acid sequence of SEQ ID NO:7 or 8, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:7 or 8 and which has the following amino acid residues at positions corresponding to positions 156, 157, 171, 172, and 205 of SEQ ID NO:7 or 8:
156: R;
157: P;
171: D;
172: T; and
205: K

4. An isolated polypeptide according to claim 1, with the following amino acid residues at positions 156, 157, 171, 172, and 205 of the head domain:
156: R;
157: P;
171: N;
172: T; and
205: K

5. An isolated polypeptide according to claim 1 or 4, which comprises an amino acid sequence of SEQ ID NO:10 or 11, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:10 or 11 and which has the following amino acid residues at positions corresponding to positions 156, 157, 171, 172, and 205 of SEQ ID NO:10 or 11:
156: R;
157: P;
171: N;
172: T; and
205: K

6. An isolated polypeptide according to claim 1, with the following amino acid residues at positions 156, 157, 171, 172, and 205 of the head domain:
156: R;
157: S;
171: N;
172: A; and
205: R

7. An isolated polypeptide according to claim 1 or 6, which comprises an amino acid sequence of SEQ ID NO:1 or 3, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:1 or 3 and which has the following amino acid residues at positions corresponding to positions 156, 157, 171, 172, and 205 of SEQ ID NO:1 or 3:
156: R;
157: S;
171: N;
172: A; and
205: R

8. An isolated polypeptide according to any preceding claim, with the following amino acid residues at positions 416 and 434 of the stem domain:
416: F; and
434: F

9. An isolated polypeptide which comprises the following amino acid sequence:
R(P/S)SFFRNVVWLIKKN(D/N)(T/A)YPTIKRSYNNTNQEDLLVLWGIHHPNDAAEQT(K/R) (SEQ ID NO:13), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:13 and which has the following amino acid residues at positions corresponding to positions 1, 2, 16, 17, and 50 of SEQ ID NO:13:
1: R;
2: P or S, preferably P;
16: D or N;
17: T or A, preferably T; and
50: K or R, preferably K.

10. An isolated polypeptide according to claim 9, with the following amino acid residues at positions 1, 2, 16, 17, and 50 of the amino acid sequence, or at positions corresponding to positions 1, 2, 16, 17, and 50 of SEQ ID NO:13:
1: R;
2: P;
16: D;
17: T; and
50: K

11. An isolated polypeptide according to claim 9, with the following amino acid residues at positions 1, 2, 16, 17, and 50 of the amino acid sequence, or at positions corresponding to positions 1, 2, 16, 17, and 50 of SEQ ID NO:13:
1: R;
2: P;
16: N;
17: T; and
50: K

12. An isolated polypeptide according to claim 9, with the following amino acid residues at positions 1, 2, 16, 17, and 50 of the amino acid sequence, or at positions corresponding to positions 1, 2, 16, 17, and 50 of SEQ ID NO:13:
1: R;
2: S;
16: N;
17: A; and
50: R

13. An isolated polypeptide which comprises an amino acid sequence of any of SEQ ID NOs:5, 9, or 12, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of any of SEQ ID NO:5, 9, or 12 and which has the following amino acid residues at positions corresponding to positions 148 and 166 of SEQ ID NO:5, 9, or 12:
148: F; and
166: F

14. An isolated polypeptide which comprises an amino acid sequence of SEQ ID NO:14, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:14.

15. An isolated polypeptide which comprises an amino acid sequence of SEQ ID NO:16, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:16.

16. An isolated polypeptide which comprises an amino acid sequence of SEQ ID NO:18, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:18.

17. An isolated nucleic acid molecule encoding a polypeptide according to any of claims 1 to 16, or an isolated nucleic acid molecule comprising a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with the nucleic acid molecule over its entire length, or the complement thereof.

18. An isolated nucleic acid molecule according to claim 17, comprising a nucleotide sequence of SEQ ID NO:2, 4, or 6, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:2, 4, or 6, over its entire length, or the complement thereof.

19. An isolated nucleic acid molecule according to claim 17, comprising a nucleotide sequence of SEQ ID NO:15, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:15, over its entire length, or the complement thereof.

20. An isolated nucleic acid molecule according to claim 17, comprising a nucleotide sequence of SEQ ID NO:17, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:17, over its entire length, or the complement thereof.

21. An isolated nucleic acid molecule according to claim 17, comprising a nucleotide sequence of SEQ ID NO:19, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:19, over its entire length, or the complement thereof.

22. A vector comprising a nucleic acid molecule of any of claims 17 to 21.

23. A vector according to claim 22, comprising a nucleic acid molecule encoding a polypeptide of any of claims 1 to 12.

24. A vector according to claim 22 or 23, comprising a nucleic acid molecule encoding a polypeptide of claim 14.

25. A vector according to any of claims 22 to 24, comprising a nucleic acid molecule encoding a polypeptide of claim 15 or 16.

26. A vector according to any of claims 22 to 25, comprising a nucleic acid molecule encoding a polypeptide which comprises an amino acid sequence of SEQ ID NO:7 or 8.

27. A vector according to any of claims 22 to 26, comprising a nucleic acid molecule encoding a polypeptide which comprises an amino acid sequence of SEQ ID NO:10 or 11.

28. A vector according to any of claims 22 to 27, comprising a nucleic acid molecule encoding a polypeptide which comprises an amino acid sequence of SEQ ID NO:1 or 3.

29. A vector according to any of claims 22 to 28, comprising a nucleic acid molecule encoding a polypeptide which comprises an amino acid sequence of SEQ ID NO:14.

30. A vector according to any of claims 22 to 29, comprising a nucleic acid molecule encoding a polypeptide which comprises an amino acid sequence of SEQ ID NO:16.

31. A vector according to any of claims 22 to 30, comprising a nucleic acid molecule encoding a polypeptide which comprises an amino acid sequence of SEQ ID NO:18.

32. A vector according to any of claims 22 to 31, which further comprises a promoter operably linked to the, or each nucleic acid molecule.

33. A vector according to claim 32, wherein the, or each promoter is for expression of a polypeptide encoded by the nucleic acid in mammalian cells.

34. A vector according to claim 33, wherein the, or each promoter is for expression of a polypeptide encoded by the nucleic acid in yeast or insect cells.

35. A vector according to any of claims 22 to 34, which is a vaccine vector.

36. A vector according to claim 35, which is a viral vaccine vector, a bacterial vaccine vector, an RNA vaccine vector, or a DNA vaccine vector.

37. An isolated cell comprising a vector of any of claims 22 to 36.

38. A fusion protein comprising a polypeptide according to any of claims 1 to 16.

39. A pharmaceutical composition comprising a polypeptide according to any of claims 1 to 16, and a pharmaceutically acceptable carrier, excipient, or diluent.

40. A pharmaceutical composition according to claim 39, comprising a polypeptide of any of claims 1 to 12.

41. A pharmaceutical composition according to claim 39 or 40, comprising a polypeptide of claim 14.

42. A pharmaceutical composition according to any of claims 39 to 41, comprising a polypeptide of claim 15 or 16.

43. A pharmaceutical composition according to any of claims 39 to 42, comprising a polypeptide which comprises an amino acid sequence of SEQ ID NO:7 or 8.

44. A pharmaceutical composition according to any of claims 39 to 43, comprising a polypeptide which comprises an amino acid sequence of SEQ ID NO:10 or 11.

45. A pharmaceutical composition according to any of claims 39 to 44, comprising a polypeptide which comprises an amino acid sequence of SEQ ID NO:1 or 3.

46. A pharmaceutical composition according to any of claims 39 to 45, comprising a polypeptide which comprises an amino acid sequence of SEQ ID NO:14.

47. A pharmaceutical composition according to any of claims 39 to 46, comprising a polypeptide which comprises an amino acid sequence of SEQ ID NO:16.

48. A pharmaceutical composition according to any of claims 39 to 47, comprising a polypeptide which comprises an amino acid sequence of SEQ ID NO:18.

49. A pharmaceutical composition comprising a nucleic acid according to any of claims 17 to 21, and a pharmaceutically acceptable carrier, excipient, or diluent.

50. A pharmaceutical composition according to claim 49, comprising a nucleic acid molecule encoding a polypeptide of any of claims 1 to 12.

51. A pharmaceutical composition according to claim 49 or 50, comprising a nucleic acid molecule encoding a polypeptide of claim 14.

52. A pharmaceutical composition according to any of claims 49 to 51, comprising a nucleic acid molecule encoding a polypeptide of claim 15 or 16.

53. A pharmaceutical composition according to any of claims 49 to 52, comprising a nucleic acid molecule encoding a polypeptide which comprises an amino acid sequence of SEQ ID NO:7 or 8.

54. A pharmaceutical composition according to any of claims 49 to 53, comprising a nucleic acid molecule encoding a polypeptide which comprises an amino acid sequence of SEQ ID NO:10 or 11.

55. A pharmaceutical composition according to any of claims 49 to 54, comprising a nucleic acid molecule encoding a polypeptide which comprises an amino acid sequence of SEQ ID NO:1 or 3.

56. A pharmaceutical composition according to any of claims 49 to 55, comprising a nucleic acid molecule encoding a polypeptide which comprises an amino acid sequence of SEQ ID NO:14.

57. A pharmaceutical composition according to any of claims 49 to 56, comprising a nucleic acid molecule encoding a polypeptide which comprises an amino acid sequence of SEQ ID NO:16.

58. A pharmaceutical composition according to any of claims 49 to 57, comprising a nucleic acid molecule encoding a polypeptide which comprises an amino acid sequence of SEQ ID NO:18.

59. A pharmaceutical composition comprising a vector according to any of claims 22 to 36, and a pharmaceutically acceptable carrier, excipient, or diluent.

60. A pharmaceutical composition according to any of claims 39 to 59, which further comprises an adjuvant for enhancing an immune response in a subject to the polypeptide, or to a polypeptide encoded by the nucleic acid, of the composition.

61. A method of inducing an immune response to an influenza virus in a subject, which comprises administering to the subject an effective amount of a polypeptide according to any of claims 1 to 16, a nucleic acid according to any of claims 17 to 21, a vector according to any of claims 22 to 36, or a pharmaceutical composition according to any of claims 39 to 60.

62. A method of immunising a subject against an influenza virus, which comprises administering to the subject an effective amount of a polypeptide according to any of claims 1 to 16, a nucleic acid according to any of claims 17 to 21, a vector according to any of claims 22 to 36, or a pharmaceutical composition according to any of claims 39 to 60.

63. A polypeptide according to any of claims 1 to 16, a nucleic acid according to any of claims 17 to 21, a vector according to any of claims 22 to 36, or a pharmaceutical composition according to any of claims 39 to 60, for use as a medicament.

64. A polypeptide according to any of claims 1 to 16, a nucleic acid according to any of claims 17 to 21, a vector according to any of claims 22 to 36, or a pharmaceutical composition according to any of claims 39 to 60, for use in the prevention, treatment, or amelioration of an influenza viral infection.

65. Use of a polypeptide according to any of claims 1 to 16, a nucleic acid according to any of claims 17 to 21, a vector according to any of claims 22 to 36, or a pharmaceutical composition according to any of claims 39 to 60, in the manufacture of a medicament for the prevention, treatment, or amelioration of an influenza viral infection.


SEQUENCE LISTING (21 sequences)

SEQ ID NO: 1 - protein, 568 aa, Artificial Sequence
  Influenza H5 designed sequence
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRSSFFRNVVWLIKKNNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 2 - DNA, 1704 bp, Artificial Sequence
  Influenza H5 designed sequence
atggaaaagattgtgctgctgctggccatcgtgtccctggtcaagagcgatcaaatctgcatcggctaccacgccaacaacagcaccgaacaggtggacaccattatggaaaagaacgtgaccgtgacacacgcccaggacatcctggaaaagacccacaacggcaagctgtgcgacctggatggcgtgaagcctctgatcctgagagattgctctgtggccggctggctgctgggcaatcctatgtgcgacgagttcatcaacgtgcccgagtggtcctatatcgtggaaaaggccaatcctgccaacgacctgtgctaccccggcaacttcaacgactacgaggaactgaaacatctgctgagccggatcaaccacttcgagaagatccagatcatccccaagtcctcttggagcgatcacgaggcctctagcggagtgtctagcgcctgtccttaccaaggcagaagcagcttcttccggaacgtcgtgtggctgatcaagaagaacaacgcttaccccaccatcaagcggagctacaacaacaccaatcaagaggacctgctggtgctgtggggcatccaccatcctaatgatgccgccgagcagacccggctgtaccagaatcctacaacctacatcagcgtgggcaccagcacactgaaccagagactggtgcctaagatcgccaccagatccaaagtgaacggccagagcggccggatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgccccaaatacgtgaagtccaacagactggtcctggccaccggcctgagaaattctccacagagagagcggcgcagaaagaagagaggcctgtttggagccattgccggctttatcgaaggcggctggcaaggcatggttgacggatggtacggctatcaccacagcaatgagcaaggctctggctacgccgccgacaaagagagcacacagaaagccatcgacggcgtgaccaacaaagtgaatagcatcatcgacaagatgaacacccagttcgaggccgtgggcagagagttcaacaacctggaaagacggatcgagaacctgaacaagaagatggaggacggcttcctggacgtgtggacctataatgccgagctgctggtcctgatggaaaacgagagaaccctggacttccacgacagcaacgtgaagaacctgtacgacaaagtgcggctccagctgcgggacaatgccaaagaactcggcaacggctgcttcgagttctaccacaagtgcgacaacgagtgcatggaaagcgtgcggaacggcacctacgactaccctcagtactctgaggaagcccggctgaagagagaagagatcagcggagtgaagctggaatccatcggcacataccagatcctgagcatctacagcaccgtggcctcttctctggccctggctattatggtggctggcctgagcctgtggatgtgctctaatggcagcctccagtgccggatctgcatc

SEQ ID NO: 3 - protein, 268 aa, Artificial Sequence
  Influenza H5 Head region designed sequence
THNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRSSFFRNVVWLIKKNNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECP

SEQ ID NO: 4 - DNA, 804 bp, Artificial Sequence
  Influenza H5 Head region designed sequence
acccacaacggcaagctgtgcgacctggatggcgtgaagcctctgatcctgagagattgctctgtggccggctggctgctgggcaatcctatgtgcgacgagttcatcaacgtgcccgagtggtcctatatcgtggaaaaggccaatcctgccaacgacctgtgctaccccggcaacttcaacgactacgaggaactgaaacatctgctgagccggatcaaccacttcgagaagatccagatcatccccaagtcctcttggagcgatcacgaggcctctagcggagtgtctagcgcctgtccttaccaaggcagaagcagcttcttccggaacgtcgtgtggctgatcaagaagaacaacgcttaccccaccatcaagcggagctacaacaacaccaatcaagaggacctgctggtgctgtggggcatccaccatcctaatgatgccgccgagcagacccggctgtaccagaatcctacaacctacatcagcgtgggcaccagcacactgaaccagagactggtgcctaagatcgccaccagatccaaagtgaacggccagagcggccggatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgcccc

SEQ ID NO: 5 - protein, 300 aa, Artificial Sequence
  Influenza H5 Stem region designed sequence
MEKIVLLLAIVSLVKSDQICIGYHANNSTEQVDTIMEKNVTVTHAQDILEKKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 6 - DNA, 900 bp, Artificial Sequence
  Influenza H5 stem region designed sequence
atggaaaagattgtgctgctgctggccatcgtgtccctggtcaagagcgatcaaatctgcatcggctaccacgccaacaacagcaccgaacaggtggacaccattatggaaaagaacgtgaccgtgacacacgcccaggacatcctggaaaagaaatacgtgaagtccaacagactggtcctggccaccggcctgagaaattctccacagagagagcggcgcagaaagaagagaggcctgtttggagccattgccggctttatcgaaggcggctggcaaggcatggttgacggatggtacggctatcaccacagcaatgagcaaggctctggctacgccgccgacaaagagagcacacagaaagccatcgacggcgtgaccaacaaagtgaatagcatcatcgacaagatgaacacccagttcgaggccgtgggcagagagttcaacaacctggaaagacggatcgagaacctgaacaagaagatggaggacggcttcctggacgtgtggacctataatgccgagctgctggtcctgatggaaaacgagagaaccctggacttccacgacagcaacgtgaagaacctgtacgacaaagtgcggctccagctgcgggacaatgccaaagaactcggcaacggctgcttcgagttctaccacaagtgcgacaacgagtgcatggaaagcgtgcggaacggcacctacgactaccctcagtactctgaggaagcccggctgaagagagaagagatcagcggagtgaagctggaatccatcggcacataccagatcctgagcatctacagcaccgtggcctcttctctggccctggctattatggtggctggcctgagcctgtggatgtgctctaatggcagcctccagtgccggatctgcatc

SEQ ID NO: 7 - protein, 568 aa, Artificial Sequence
  Influenza H5 designed sequence
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRPSFFRNVVWLIKKNDTYPTIKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 8 - protein, 268 aa, Artificial Sequence
  Influenza H5 head region designed sequence
THNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRPSFFRNVVWLIKKNDTYPTIKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECP

SEQ ID NO: 9 - protein, 300 aa, Artificial Sequence; identical to SEQ ID NO: 5
  Influenza H5 stem region designed sequence

SEQ ID NO: 10 - protein, 568 aa, Artificial Sequence
  Influenza H5 designed sequence
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRPSFFRNVVWLIKKNNTYPTIKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 11 - protein, 268 aa, Artificial Sequence
  Influenza H5 head region designed sequence
THNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRPSFFRNVVWLIKKNNTYPTIKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECP

SEQ ID NO: 12 - protein, 300 aa, Artificial Sequence; identical to SEQ ID NO: 5
  Influenza H5 stem region designed sequence

SEQ ID NO: 13 - protein, 50 aa, Artificial Sequence
  Influenza H5 head domain fragment designed sequence
  MISC_FEATURE (2)..(2): Residue 2 is P or S
  MISC_FEATURE (16)..(16): Residue 16 is D or N
  MISC_FEATURE (17)..(17): Residue 17 is T or A
  MISC_FEATURE (50)..(50): Residue 50 is K or R
RXSFFRNVVWLIKKNXXYPTIKRSYNNTNQEDLLVLWGIHHPNDAAEQTX

SEQ ID NO: 14 - protein, 97 aa, Artificial Sequence
  Influenza M2 designed sequence
MSLLTEVETPTRNGWECRCSDSSDPLVIAASIIGILHLILWILDRLFFKCIYRRLKYGLKRGPSTEGVPESMREEYRQKQQSAVDVDDGHFVNIELE

SEQ ID NO: 15 - DNA, 291 bp, Artificial Sequence
  Influenza M2 designed sequence
atgtctctgctgaccgaggtggaaacccctaccagaaatggctgggagtgcagatgcagcgacagcagcgatcctctggttatcgccgccagcatcatcggcatcctgcacctgatcctgtggatcctggaccggctgttcttcaagtgcatctaccggcggctgaagtacggcctgaagagaggcccttctacagagggcgtgcccgagagcatgcgggaagagtacagacagaaacagcagagcgccgtggacgtggacgatggccacttcgtgaacatcgagctggaa

SEQ ID NO: 16 - protein, 469 aa, Artificial Sequence
  Influenza NA designed sequence
MNPNQKIITIGSICMVVGIISLILQIGNIISIWVSHSIQTGNQNQPETCNQSIITYENNTWVNQTYVNISNTNFVAEQAVASVALAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACINGSCFTIMTDGPSNGQASYKIFKIEKGKVVKSVELNAPNYHYEECSCYPDAGEVMCVCRDNWHGSNRPWVSFNQNLEYQIGYICSGVFGDNPRPNDGTGSCGPVSSNGAYGVKGFSFKYGKGVWIGRTKSTSSRSGFEMIWDPNGWTETDSSFSVKQDIVAITDWSGYSGSFVQHPELTGLDCMRPCFWVELIRGRPKENTIWTSGSSISFCGVNSDTVGWSWPDGAELPFTIDK

SEQ ID NO: 17 - DNA, 1407 bp, Artificial Sequence
  Influenza NA designed sequence
atgaatccaaatcagaaaataataaccattgggtcaatctgtatggtagttggaataatcagcctaatattacaaattgggaacataatctcaatatgggttagccattcaattcagactggaaatcaaaaccaacctgaaacatgcaaccaaagcatcattacttatgaaaacaacacttgggtgaatcaaacatatgttaacatcagcaataccaattttgttgctgaacaggctgtagcttcagtggcattagcgggcaattcctctctctgccccattagtgggtgggctatatacagcaaggacaatggcataaggattggttccaagggagatgtatttgtcataagagagccattcatttcatgctcccacttggaatgcaggaccttttttctgactcaaggagccttgttgaatgacaaacattccaatggaaccgttaaagacagaagcccctacagaaccttaatgagctgtcctgttggtgaggctccctctccatacaattcaaggtttgagtcggttgcttggtcagcaagtgcttgccatgatggcattagctggttgacaattggaatttccgggccagacaatggggcagtggctgtattgaaatacaatggcataataacagacactatcaaaagttggagaaacaacatattgaggacacaagagtctgaatgtgcctgcataaatggttcttgctttactataatgaccgatggaccaagtaatgggcaggcctcatacaagattttcaagatagagaaggggaaggtagtcaaatcagtcgagttgaatgcccctaattaccactacgaggaatgttcctgttatcctgatgctggcgaagtaatgtgtgtgtgcagggataattggcatggttcgaatcgaccatgggtgtctttcaatcaaaatctggagtatcaaataggatacatatgcagtggggttttcggagacaatccacgccccaatgatggaacaggcagctgtggtccagtgtcttctaatggagcatatggagtaaagggattttcatttaagtacggcaagggtgtttggatagggagaactaagagcactagttccaggagtggatttgagatgatttgggatcccaatggatggacagagacagatagtagtttctcagtgaagcaagatattgtagcaataactgattggtcaggatatagcgggagttttgtccaacatccagaattaacagggctggactgcatgaggccttgcttctgggttgaactaatcagaggacggcctaaggagaacacaatctggactagtgggagcagcatttccttctgtggtgtaaatagcgacactgtgggttggtcttggccagacggtgctgagttgccattcaccattgacaag

SEQ ID NO: 18 - protein, 469 aa, Artificial Sequence
  Influenza NA designed sequence
MNPNQKIITIGSICMVVGIISLILQIGNIISIWVSHSIQTGNQNHPETCNQSIITYENNTWVNQTYVNISNTNVVAGQDATSVILAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGMGWLTIGISGPDNGAVAVLKYNGIITDTIKSWRNNILRTQESECACVNGSCFTIMTDGPSNGQASYKIFKIEKGKVIKSIELNAPNYHYEECSCYPDTGKVMCVCRDNWHGSNRPWVSFDQNLDYQIGYICSGVFGDNPRPNDGTGSCGPVSSNGANGVKGFSFRYGNGVWIGRTKSTSSRSGFEMIWDPNGWTETDSSFSVKQDIVAITDWSGYSGSFVQHPELTGLDCMRPCFWVELIRGQPKENTIWTSGSSISFCGVNSDTVGWSWPDGAELPFTIDK

SEQ ID NO: 19 - DNA, 1407 bp, Artificial Sequence
  Influenza NA designed sequence
atgaatccaaatcaaaaaataataaccattgggtcaatctgtatggtagttggaataattagcctaatattgcaaatagggaatataatctcaatatgggttagccattcaattcaaactggaaatcaaaaccatcctgaaacatgcaaccaaagcatcattacctatgaaaataacacctgggtgaatcaaacatatgttaacattagcaatactaacgttgttgctggacaggatgcaacttcagtgatattagccggcaattcctctctttgccccatcagtgggtgggctatatacagcaaagacaatggcataagaattggttccaaaggagacgtttttgtcataagagagccatttatttcatgctctcacttggaatgcaggaccttttttctgactcaaggcgccttgctgaatgacaagcattcaaatgggaccgtcaaggacagaagcccctatagaaccttaatgagctgccctgttggtgaagctccgtctccgtacaattcaaggttcgaatcggttgcttggtcagcaagtgcatgccatgatggcatgggctggctaacaatcggaatttccggtccagataatggagcagtggctgtattaaaatacaatggtataataacagacaccatcaaaagttggaggaacaacatattgagaacgcaagagtctgaatgtgcctgtgtaaatggttcatgttttactataatgaccgatggcccaagtaatgggcaggcctcgtacaaaattttcaagatagagaaggggaaggttattaaatcaattgagttgaatgcacctaattaccactacgaggaatgttcctgttaccctgatacaggtaaagtgatgtgtgtgtgcagagacaattggcatggttcgaatcgaccatgggtgtctttcgatcaaaatctggattatcaaataggatacatctgcagtggggttttcggtgacaatccgcgtcccaatgatggaacaggcagctgtggtccagtgtcttctaatggagcaaatggagtaaagggattttcatttaggtatggtaatggtgtttggataggaagaactaaaagtaccagttccagaagcgggtttgagatgatttgggatcctaatggatggacagagactgatagtagtttctctgtgaaacaagatattgtagcaataactgattggtcagggtacagcgggagtttcgttcaacatcctgagctaacagggctggactgcatgaggccttgcttctgggttgaattaatcaggggacaacctaaagagaacacaatctggactagtgggagcagcatttccttttgtggcgtaaatagtgatactgtaggttggtcttggccagacggtgctgagttgccattcaccattgacaag

SEQ ID NO: 20 - DNA, 91 bp, Artificial Sequence
  pEVAC Multiple Cloning Site sequence
acagactgttcctttccatgggtcttttctgcagtcaccgtcggtaccgtcgacacgtgtgatcatctagaggatccgcggccgcagatct

SEQ ID NO: 21 - DNA, 4405 bp, Artificial Sequence
  pEVAC sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgtcgacacgtgtgatcatctagaggatccgcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc